(19) United States
(12) Patent Application Publication
(10) Pub. No.: US 2022/0161234 A1
(43) Pub. Date: May 26, 2022

(54) CARBON DOTS, METHODS OF MANUFACTURE THEREOF, AND USES THEREOF IN THE PRODUCTION OF BIOFUEL

(71) Applicant: VALORBEC, SEC, Montréal (CA)

(72) Inventors: Alexia MACINA, Montréal (CA); Tayline Viana DE MEDEIROS, Montréal (CA); Rafik NACCACHE, Laval (CA); Miguel Mariano RODRIGUEZ, Saint-Laurent (CA)

(73) Assignee: VALORBEC, SEC, Montréal, QC (CA)

(21) Appl. No.: 17/611,568

(22) PCT Filed: May 19, 2020
(86) PCT No.: PCT/CA2020/050670

§ 371 Date: Nov. 16, 2021

Related U.S. Application Data
(60) Provisional application No. 62/849,576, filed on May 17, 2019.
(60) Provisional application No. 62/868,677, filed on Jun. 28, 2019.
(60) Provisional application No. 62/896,011, filed on Sep. 5, 2019.

(51) Int. Cl. B01J 21/18; C07C 235/12; C07C 67/03; C10L 1/02

(52) U.S. Cl. CPC B01J 21/18; C07C 235/12; C10L 2200/0476; C10L 1/026; C07C 67/03

(57) ABSTRACT

The present disclosure relates to carbon dots, uses thereof and methods of manufacture thereof. For example, such carbon dots can be used in the production of biofuels such as biodiesel. For example, these carbon dots can be used as catalysts in transesterification reactions. For example, these carbon dots can be glycine-citric acid carbon dots, amine-passivated carbon dots, or combinations thereof.

### CROSS-REFERENCE TO RELATED APPLICATIONS

The present application claims priority to U.S. application No. 62/849,576 filed on May 17, 2019; U.S. application No. 62/868,677 filed on Jun. 28, 2019; and U.S. application No. 62/896,011 filed on Sep. 5, 2019. These documents are hereby incorporated by reference in their entirety.

### FIELD OF THE DISCLOSURE

The present disclosure generally relates to catalysts in the production of biofuel and more particularly to carbon dots, methods of manufacture thereof, and uses thereof in the production of biofuel.

### BACKGROUND OF THE DISCLOSURE

Rapid technological progress and globalization efforts have dictated the urgency in fulfilling the world's ever-increasing energy demands. According to a 2018 report by the International Energy Agency, global energy demands will grow a staggering 40% by the year 2040.¹ Traditional sources of energy stem from non-renewable fossil fuels which include coal, natural gas and oil. Fossil fuels are generated over millions of years through long term anaerobic decomposition of organic matter in the earth's crust.² The overall consumption of these fossil fuels increased more than 6-fold since 1950.³ It follows that these energy reservoirs cannot be replenished as rapidly as they are being consumed. Moreover, with a persistent increase in demand and consumption of fossil fuels, an abrupt increase in CO2 and other greenhouse gas emissions have been observed.^{4,5} The unsustainable and finite nature of fossil fuel reservoirs along with the tremendous negative environmental impact, associated with its extraction and burning, has driven the urgent need to find alternative sources of renewable energy. Indeed, significant financial resources and research efforts are being invested into the creation of alternative forms of energy which have the potential to meet future energy demands in a cost efficient and environmentally sustainable manner, while being as energy efficient as conventional fossil fuels.

Biodiesel fuel production remains one of the most promising alternatives to non-renewable fossil fuels and has been touted to replace conventional oil and diesel. Several advantages are attributed to the production of biodiesel, including its high biodegradability and minimal toxicity while maintaining similar physico-chemical properties to conventional diesel.^{6,7} Chemically speaking, biodiesel is composed of a mixture of long chain fatty acid methyl esters arising from the conversion of triglycerides through a transesterification reaction.⁸ Biodiesel can be obtained through the conversion of vegetable oil in the presence of an alcohol (e.g. methanol) under basic or acidic conditions. In this case, a homogenous base or acid catalyst such as KOH⁹⁻¹¹, or H₂SO₄,¹²⁻¹⁴, respectively, are required to catalyse the formation of methyl esters from the fatty acid chains in the vegetable oil. Currently, industry scale biodiesel is produced using a base catalyst such as KOH or NaOH.¹⁵ High conversion yields (>98%) can be achieved using a small percentage of the base catalyst, usually amounting to 0.5-1 wt % the vegetable oil precursor.¹⁴ While homogeneous catalysts are reliable and relatively inexpensive, their use comes with several drawbacks including: (i) saponification which greatly hinders the formation of biodiesel, (ii) difficult and costly purification and separation of the biodiesel from the catalyst, and (iii) the inability for catalyst regeneration.^{7,16} Such drawbacks have limited the widespread adoption and production of biodiesel on a global scale. It is in this regard that there has been significant efforts focused on developing a heterogeneous catalyst alternative. Heterogeneous catalysts can effectively address the shortcomings of their homogeneous counterparts. They do not induce saponification and can be regenerated and re-used over multiple catalytic cycles. Indeed, many materials have been reported as promising heterogeneous catalysts for biodiesel production, including basic metal oxides such as CaO¹⁷ and SrO¹⁸, mesoporous supported materials such as silica¹⁹, zeolites²⁰⁻²² and MCM-41²³⁻²⁵, as well as enzymes,²⁶⁻²⁹ among others. While catalytically efficient, these heterogeneous catalysts require a high loading (up to 15 wt %), are expensive and can be prone to metal leaching.

Carbon-based materials present an interesting alternative and have also been reported as efficient heterogeneous catalysts for biodiesel production including acid and basic-activated carbon, sulfonated carbon nanotubes, and supported carbon-materials. These carbon materials present easier and inexpensive synthetic routes when compared to the other heterogeneous catalysts, high thermal stability, as well as a metal-free composition.^{8,30} Among the carbon-allotropes family, carbon dots (CDs) have been shown to be a promising candidate in several catalytic applications.³¹⁻³³ Ranging from 1-10 nm in size, they are comprised of mostly sp² carbons along with hydrogen, oxygen and nitrogen. The dots can be synthesized from a wide variety of carbon containing precursors all of which dictate the resultant surface functional groups. Their versatile chemistry and stability allow for the possibility to tune the surface physico-chemical properties targeting catalytic applications including the transesterification of biodiesel.

### SUMMARY OF THE DISCLOSURE

A first aspect disclosed herein relates to a use of carbon dot to catalyze a transesterification reaction.

Another aspect disclosed herein is a use of carbon dot to carry out a transesterification reaction of an oil into biofuel.

In another aspect there is provided a use of carbon dot as heterogeneous catalyst to catalyze a transesterification reaction.

A further aspect relates to a use of carbon dot as a heterogeneous catalyst in the preparation of a biofuel.

Also provided herein in a further aspect is a carbon dot obtained by reacting together:
a carboxylic acid and/or a carboxylate; and
a base;
wherein said reacting is carried out in a temperature mediated reaction.

Also provided herein in a further aspect is a carbon dot obtained by reacting together:
a carboxylic acid and/or a carboxylate;
a base; and
a primary amine and/or a secondary amine,
wherein said reacting is carried out in a temperature mediated reaction.

Another aspect relates to a carbon dot obtained in a temperature mediated reaction by reacting together:
a carboxylic acid and/or a carboxylate;
a base; and
a primary amine and/or a secondary amine,
wherein said reacting is carried out in a temperature mediated reaction.

Another aspect relates to a carbon dot obtained in a temperature mediated reaction by reacting together:
a carboxylic acid and/or a carboxylate; and
a base;
wherein said reacting is carried out in a temperature mediated reaction.

Another aspect relates to a carbon dot obtained by reacting together
a carboxylic acid and/or a carboxylate; and
a primary amine and/or a secondary amine,
wherein said reacting is carried out in a temperature mediated reaction.

A carbon dot obtained in a temperature mediated reaction by reacting together:
a carboxylic acid and/or a carboxylate; and
a primary amine and/or a secondary amine,
wherein said reacting is carried out in a temperature mediated reaction.

In another aspect, there is provided a method of preparing a carbon dot comprising contacting a carboxylic acid and/or a carboxylate, a base, and a primary and/or a secondary amine in a temperature mediated reaction.

In another aspect, there is provided a method of preparing a carbon dot comprising contacting a carboxylic acid and/or a carboxylate and a primary and/or a secondary amine in a temperature mediated reaction.

In another aspect, there is provided an amine-passivated citric acid carbon dot.

In another aspect, there is provided an amine-passivated citric acid carbon dot having a characteristics as shown in at least one of FIG. 11(a), FIG. 11(b), FIG. 11(c), FIG. 16(a), FIG. 16(b), FIG. 16(c), and FIG. 16(d).

In another aspect, there is provided an amine-passivated citric acid carbon dot as described in the present description and FIGS. 11 and 16.

In another aspect, there is provided a glycine-citric acid carbon dot.

In still another aspect, there is provided a glycine-citric acid carbon dot having a characteristic as shown in at least one of FIG. 1(a), FIG. 1(b), FIG. 1(c), FIG. 1(d), FIG. 1(e) and FIG. 1(f).

A further aspect relates to a glycine-citric acid carbon dot having a characteristic as shown in at least one of FIG. 1(a), FIG. 1(b), FIG. 1(c), FIG. 1(d), FIG. 1(e), FIG. 1(f) and FIG. 5.

A further aspect relates to a glycine-citric acid carbon dot as described in the present description and FIGS. 1-8.

Also disclosed herein in an aspect is a method of carrying out a transesterification reaction, the method comprising reacting an ester with an alcohol in the presence of a catalyst comprising carbon dot.

Yet another aspect relates to a method of preparing a biofuel comprising reacting an oil and an alcohol in the presence of a carbon dot catalyst.

In another aspect there is provided a method of preparing a biofuel comprising catalyzing a transesterification reaction between an oil and an alcohol by reacting the oil and the alcohol in the presence of a carbon dot catalyst.

### BRIEF DESCRIPTION OF THE FIGURES

Further features and advantages of the disclosure will become more readily apparent from the following description of specific embodiments as illustrated by way of examples in the appended figures wherein:

FIG. 1(a) is a TEM image of a 1 mg·mL⁻¹ dispersion of GlyCDs dispersed in MeOH. The TEM images show quasi-spherical dots with a calculated particle size of 10.9±2.8 nm and a Gaussian particle size distribution ranging from 6 to 16 nm. FIG. 1(b) shows FT-IR spectra of the citric acid revealing characteristic vibrations for alcohol and carboxyl groups. The FT-IR spectrum of glycine precursor reveals amino acid functional groups such as amine and carboxyl moieties. The FT-IR spectrum of the GlyCDs reveal amine and carboxy functional groups. FIGS. 1(c), 1(d) 1(e) and 1(f) display the results of an XPS survey scan of GlyCDs revealing five binding energies ascribed to Na1s, O1s, Na KLL, N1s and C1s. Deconvoluted spectra of HR-XPS shows (d) N1s binding energy maxima at 399.63 eV; (e) C1s binding energy maxima at 286.94 eV; (f) O1s binding energy maxima at 532.39 eV.

FIG. 2(a) is a schematic of a transesterification reaction. FIG. 2(b) is a calibration curve constructed using known amounts of methyl ester canola oil, used to determine biodiesel conversion. FIG. 2(c) shows ¹H NMR spectra of canola oil and biodiesel showing the three significant peaks at 4.30 ppm and 2.30 ppm pertaining to triglyceride backbone protons and methylene protons adjacent to the carbonyl in canola oil and at 3.66 ppm pertaining to the methoxy protons in biodiesel. FIG. 2(d) is a FT-IR analysis of canola oil and biodiesel. The major stretches associated to biodiesel are the asymmetric and symmetric stretches at 1443 cm⁻¹ and 1196 cm⁻¹ attributed to the —CH₃ and O—CH₃ of the methyl esters formed in biodiesel.

FIGS. 3(a), 3(b) and 3(c) are bar graphs showing the percent biodiesel conversion from canola oil using GlyCDs when varying (a) % wt GlyCDs catalyst, (b) reaction temperature, and (c) reaction time. FIG. 3(d) is a bar graph showing the biodiesel conversion from canola oil using GlyCDs when according to the number of reactions, demonstrating the reusability and stability of GlyCDs. Reaction conditions were kept constant for each cycle. FIG. 3(e) shows FT-IR spectra of glycerol-GlyCD bottom layer after transesterification reaction, glycerol-GlyCDs bottom layer after transesterification reaction two and subjection to hexanes organic wash, and crude glycerol. FIG. 3(f) is a reaction scheme of the proposed transesterification mechanism GlyCDs as catalysts.

FIG. 4 is a graph showing absorbance and fluorescence spectra of glycine-citric acid CDs. The absorbance spectrum presents two bands with the first attributed to the π→π** transition corresponding to alkene and aromatic sp² domains (black) and the second to the n→π* transition associated with functional groups, such as carboxyl and amides (red). PL spectrum (inset, blue) shows a maximum λₑₘ at 400 nm following ×ₑₓ=300 nm.

FIG. 5 is a thermogravimetric analysis of GlyCDs showing a three-step decomposition weight loss pattern. The first weight loss (9.2%) occurs at 140° C. and can be attributed to water loss. The second weight loss (36.2%) occurs at 385° C., and is related to decomposition of the surface moieties. Finally, the third weight loss (42.6%) occurs at 795° C. and is associated with the decomposition of the carbon core.

FIG. 6 is a graph showing transesterification reaction using 1% GlyCDs at 2 and 3 hour reaction times and 1:60 and 1:18 oil to methanol ratios. At lower methanol concentrations, a reaction time of 3 hours is required to ensure conversion at ˜97%.

FIG. 7 Negative controls to ensure the biodiesel formation stems from the GlyCD catalysts. Protonated GlyCDs (red) and the reaction of methanol and canola oil (black), in the absence of the CD catalyst, did not result in any significant biodiesel conversion.

FIG. 8 is an image of a 1% agarose gel electrophoresis performed at pH 7.4 for the GlyCDs at two different concentrations. The result shows the GlyCDs migrating towards the positively charged anode indicating that they are negatively charged.

FIG. 9 shows a schematic of an example of the synthesis of amine-passivated CDs.

FIG. 10 shows a schematic of an example of the transesterification of canola oil to biodiesel using CD catalysts.

FIG. 11 shows the physical properties of deprotonated 750 mM DT3-CDs; (a) TEM image of a 1 mg mL⁻¹ dispersion of amine-passivated CDs dispersed in MeOH, showing quasi-spherical dots with a calculated particle size of 13.2±3.1 nm and a Gaussian particle size distribution ranging from 6 to 26 nm; (b) XRD diffraction pattern showing an amorphous halo in the range 10-80° 2θ; (c) TGA thermogram showing a four-step decomposition profile and confirming the high thermal stability of synthesized CDs.

FIG. 12 shows XRD diffractograms for all synthesized protonated amine-passivated CDs. Diffraction patterns for all synthesized CDs exhibit an amorphous halo in the range 10-80° 28 and lack any crystalline reflections, confirming that the prepared CDs were amorphous in nature.

FIG. 13 shows differential thermogravimetry curves that show four-step decomposition profiles for deprotonated 125-750 mM DT3-CDs (panel (a)) and deprotonated 375 mM amine-passivated CDs 9panel (b)). In panel (c), TGA thermograms shows a four-step decomposition profile and confirming a high thermal stability of all synthesized amine-passivated CDs.

FIG. 14 shows aqueous 750 mM DT3-CD dispersions exhibiting blue fluorescence upon excitation at 365 nm.

FIG. 15 shows optical properties of amine-passivated CDs. Panels (a) and (c) are UV-Vis and fluorescence spectra of aqueously dispersed DT3-CDs at 210° C. using 125-750 mM, respectively. Panels (b) and (d) are UV-Vis and fluorescence spectra of aqueously-dispersed amine-passivated CDs synthesized at 210° C. using 375 mM of different amine-passivating agents, respectively.

FIG. 16 shows in panels (a,b) FT-IR spectra of protonated and deprotonated DT3-CDs synthesized at 210° C. using 125-750 mM DT3; and in panels (c,d) FT-IR spectra of protonated and deprotonated amine-passivated CDs synthesized at 210° C. using 375 mM of different amine-passivating agents. All FT-IR spectra reveal characteristic vibrations for amide and carboxyl moieties. confirmed by the carbonyl stretching at ˜1645 cm⁻¹ and ˜1695 cm⁻¹, respectively. Furthermore, all amine-passivated CDs exhibit 0-H and N—H stretching at ˜3380 cm⁻¹ and ˜3255 cm⁻¹, aromatic C═C stretching at ˜1540 cm⁻¹ and symmetric carboxylate stretching at ˜1360 cm⁻¹.

FIG. 17 shows in panel (a)¹H NMR assignments and spectra for canola oil and biodiesel, revealing the characteristic peaks at 4.30 ppm ascribed to the triglyceride backbone protons in canola oil and at 3.66 ppm assigned to the methoxy protons in biodiesel. The triplet at 2.30 ppm representing the α-methylene group in triglyceride derivatives was used as a basis point for calculations. In panel (b), FT-IR spectra of canola oil and biodiesel are shown, revealing the characteristic bands at ˜1450 cm⁻¹ and 1196 cm⁻¹ pertaining to the asymmetric methyl stretching and methoxy stretching, respectively, in biodiesel.

FIG. 18 shows ¹H NMR spectra of canola converted to biodiesel using (a) protonated and (b) deprotonated 750 mM DT3-CDs at a catalyst loading of 10 wt % and an oil to methanol ratio of 1:72. Reactions were heated at 150° C. for 3 hours. The ¹H NMR spectrum of a protonated 750 mM DT3-CDs catalysed transesterification reaction (a) illustrates the negligible biodiesel conversion yield achieved by CDs in their protonated form, demonstrated by the lack of the methoxy singlet at 3.66 ppm. The ¹H NMR spectrum of a deprotonated 750 mM DT3-CDs catalysed transesterification reaction (b) illustrates the drastic increase in biodiesel conversion yield achieved by CDs in their deprotonated form (98%), demonstrated by the intense methoxy singlet at 3.66 ppm.

FIG. 19 shows percent biodiesel conversion of deprotonated 750 mM DT3-CDs catalysed transesterification reactions when varying (a) % wt catalyst loading, (b) oil to methanol ratios, (c) temperature, and (d) reaction time; (e) Reusability and stability studies of deprotonated 750 mM DT3-CDs. Reaction conditions were kept constant for each cycle.

FIG. 20 shows percent biodiesel conversion for transesterification reactions catalysed by (a) deprotonated DT3-CDs at 210° C. using 125-750 mM DT3, and (b) deprotonated amine-passivated CDs synthesized at 210° C. using 375 mM of different amine-passivating agents; (c) Proposed transesterification mechanism using amine-passivated CD catalysts.

### DETAILED DESCRIPTION OF THE DISCLOSURE

Various embodiments of the present disclosure are hereby provided in a non-limiting manner.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

Carbon dots (CDs) can be synthesized from an abundance of different molecular precursors and as a result, surface functional groups that partake in catalytic reactions can be easy tailored. In fact, it is possible to mimic homogeneous catalysis systems by designing CDs that bear alcohol and carboxylate moieties on their surface. This would potentially impart them with the same catalytic efficiency of homogeneous catalysts, require low catalyst loading, allow for recovery and reusability without the possibility of metal leaching.

Accordingly, the design of a heterogeneous CD catalyst prepared from citric acid and glycine for the transesterification reaction of canola oil to produce biodiesel is disclosed herein. The surface physical properties and chemistry of the CDs have been characterized in order to glean an understanding of the functional groups and the surface chemistry of these dots. The transesterification of canola oil to biodiesel through a quantitative ¹H NMR technique has been studied. Furthermore, the effects of the transesterification reaction parameters including temperature, methanol content, catalyst loading and time on the transesterification reaction were investigated. Lastly, the recovery and reusability of the catalyst for multiple catalytic cycles and the impact on conversion efficiency is demonstrated herein.

For example, the transesterification is carried out by using carbon dots as a heterogeneous catalyst.

For example, the oil is a vegetable oil, a waste oil, a grease trap oil, animal fat, distillers oils, recycled oil or mixtures thereof.

For example, the oil is canola oil, soybean oil, corn oil, rapeseed oil, sunflower oil, safflower oil, peanut oil, cottonseed oil, coconut oil, palm oil, rice bran oil, or mixtures thereof.

The present disclosure generally relates to catalysts in the production of biofuel and more particularly to carbon dots, methods of manufacture thereof, and uses thereof in the production of biofuel.

For example, the transesterification is carried out at a temperature of about 50° C. to about 250° C. For example, the transesterification is carried out at a temperature of about 100° C. to about 200° C. For example, the transesterification is carried out at a temperature of about 125° C. to about 175° C. For example, the transesterification is carried out at a temperature of about 130° C. to about 170° C. For example, the transesterification is carried out at a temperature of about 140° C. to about 160° C. For example, the transesterification is carried out at a temperature of about 150° C.

For example, the transesterification is carried out with a catalyst loading of about 0.1 to about 40 wt %. For example, the transesterification is carried out with a catalyst loading of about 0.1 to about 30 wt %. For example, the transesterification is carried out with a catalyst loading of about 0.1 to about 20 wt %. For example, the transesterification is carried out with a catalyst loading of about 0.2 to about 20 wt %.For example, the transesterification is carried out with a catalyst loading of about 0.5 to about 10 wt %. For example, the transesterification is carried out with a catalyst loading of about 0.5 to about 8 wt %. For example, the transesterification is carried out with a catalyst loading of about 0.5 to about 6 wt %. For example, the transesterification is carried out with a catalyst loading of about 0.5 to about 4 wt %. For example, the transesterification is carried out with a catalyst loading of about 0.5 to about 2 wt %. For example, the transesterification is carried out with a catalyst loading of about 0.8 to about 1.5 wt %. For example, the transesterification is carried out with a catalyst loading of about 1.0 wt %.

For example, the transesterification is carried out in an oil:alcohol ratio of about 1:5 to about 1:200. For example, the transesterification is carried out in an oil:alcohol ratio of about 1:5 to about 1:100. For example, the transesterification is carried out in an oil:alcohol ratio of about 1:5 to about 1:80. For example, the transesterification is carried out in an oil:alcohol ratio of about 1:10 to about 1:80. For example, the transesterification is carried out in an oil:alcohol ratio of about 1:20 to about 1:80. For example, the transesterification is carried out in an oil:alcohol ratio of about 1:30 to about 1:80. For example, the transesterification is carried out in an oil:alcohol ratio of about 1:40 to about 1:80. For example, the transesterification is carried out in an oil:alcohol ratio of about 1:15 to about 1:35. For example, the transesterification is carried out in an oil:alcohol ratio of about 1:25 to about 1:30. For example, the transesterification is carried out in an oil:alcohol ratio of about 1:27. For example, the transesterification is carried out in an oil:alcohol ratio of about 1:60.

For example, the alcohol is chosen from methanol, ethanol, butanol, propanol, iso-propanol, and mixtures thereof.

For example, the carbon dots are selected from glycine-citric acid carbon dots (GlyCDs), amine-passivated citric acid carbon dots (amine-passivated CDs), and combinations thereof.

For example, biofuel is biodiesel.

Another aspect of the disclosure relates to a carbon dot obtained according to methods described herein.

In another aspect, there is provided a method of preparing a carbon dot comprising contacting a carboxylic acid and/or a carboxylate, a base, and a primary and/or a secondary amine in a temperature mediated reaction.

In another aspect, there is provided a method of preparing a carbon dot comprising contacting a carboxylic acid and/or a carboxylate and a primary and/or a secondary amine in a temperature mediated reaction.

For example the primary amine is an amino acid. For example, the secondary amine is an amino acid.

For example, the primary and/or said secondary amine is chosen from ethylene diamine, diethylene triamine, triethylene tetramine, tetraethylene pentamine, and pentaethylene hexamine.

For example, the amino acid is chosen from alanine, aspartic acid, glutamic acid, phenyalanine, glycine, histidine, isoleucine, lysine, leucine, methionine, asparagine, pyrrolysine, proline, glutamine, arginine, serine, threonine, selenocysteine, valine, tryptophan, tyrosine, and mixtures thereof.

For example, the amino acid is glycine.

For example, the carbon dot is substantially metal free. For example, the carbon dot excludes the presence of a metal-doped carbon dots. For example, the carbon dot excludes the presence of metal containing-carbon dots.

For example, the metal is chosen from cadmium, lead, zinc, gold, silver, iron, manganese, cobalt, nickel, gallium and tin.

For example, the carbon dot is synthesized using a bottom up approach, optionally using electrochemical synthesis, combustion/thermal reaction, microwave reactions and ultrasonic reactions,

For example, the temperature mediated reaction is carried out in a hydrothermal reactor or in a microwave reactor.

For example, the carbon dot is synthesized using a top down approach, optionally using laser ablation, attrition, milling and/or arc discharge.

For example, the base is sodium hydroxide (NaOH), potassium hydroxide (KOH), ammonium hydroxide (NH₄OH), lithium hydroxide (LiOH), calcium hydroxide (Ca(OH)₂) or barium hydroxide (Ba(OH)₂). For example, the base is NaOH.

For example, the carboxylic acid is citric acid, ascorbic acid, malic acid, malonic acid, succinic acid or glutaric acid. For example, the carboxylic acid is citric acid.

For example, the reaction is carried out at a temperature of about 180° C. to about 240° C. For example, the reaction is carried out at a temperature of about 200° C. to about 220° C.

For example, the reaction is carried out in water. For example, the water is deionized water.

For example, the reaction comprises stirring said carboxylic acid and/or carboxylate, said base and said primary amine and/or secondary amine.

For example, the stirring is carried out at about 400 rpm to 700 rpm. For example, the stirring is carried out at about 500 rpm to about 660 rpm.

For example, the reaction is carried out for a duration of about 1 hours to about 5 hours. For example, the reaction is carried out for a duration of about 3 to about 5 hours, optionally about 4 hours.

Once the carbon dots are synthesized, additional steps may be carried out to further purify the carbon dots and remove impurities. For example, the carbon dot composition is further dialyzed against water after the reaction for removing unreacted precursors and fluorophores. For example, the precipitate obtained after dialysis is further washed. For example, the washed carbon dot composition is centrifuged to obtain a carbon dot precipitate. For example, the centrifuging is carried out at about 10,000×g for about 10 minutes. For example, the carbon dot precipitate is repeatedly washed and centrifuged.

For example, the carbon dot precipitate is further dried to obtain the carbon dot. For example, the carbon dot precipitate is dried at about 70° C. to about 100° C. For example, the carbon dot precipitate is dried at about 80° C. to about 90° C. For example, the carbon dot precipitate is dried for about 24 hours to about 72 hours. For example, the carbon dot precipitate is dried for about 48 hours. For example, the carbon dot precipitate is oven-dried.

For example, the glycine-citric acid carbon dot has an average particle size distribution of about 1 nm to about 15 nm. For example, the glycine-citric acid carbon dot has an average particle size distribution of about 5 nm to about 15 nm. For example, the glycine-citric acid carbon dot has an average particle size distribution of up to about 15 nm. For example, the glycine-citric acid carbon dot has an average particle size distribution of less than about 50 nm.

For example, the reaction of said carboxylic acid and/or carboxylate and a primary amine and/or a secondary amine comprises stirring said carboxylic acid and/or carboxylate and a primary amine and/or a secondary amine.

For example, the reaction of said carboxylic acid and/or carboxylate and a primary amine and/or a secondary amine is carried out for about 5 minutes to about 1 hour. For example, the reaction of said carboxylic acid and/or carboxylate and a primary amine and/or a secondary amine is carried out for a duration of about 5 minutes to about 30 minutes. For example, the reaction of said carboxylic acid and/or carboxylate and a primary amine and/or a secondary amine is carried out for a duration of about 5 minutes to about 15 minutes. For example, the reaction of said carboxylic acid and/or carboxylate and a primary amine and/or a secondary amine is carried out for a duration of about 10 minutes.

In some embodiments, the reaction of the reaction of said carboxylic acid and/or carboxylate and a primary amine and/or a secondary amine produces said carbon dot in a carbon dot composition. In some embodiments, said carbon dot composition comprises said carbon dot, unreacted precursors, fluorophores and/or impurities. Once said carbon dot is synthesized, additional steps can be performed to purify said carbon dot. For example, said carbon dot composition obtained after said reaction is dialyzed against water for removing unreacted precursors, fluorophores, and/or polar impurities. For example, the dialyzed carbon dot composition is washed to remove impurities. For example, the washed carbon dot composition is centrifuged to obtain a carbon dot precipitate. For example, said centrifugation is carried out at about 10,000×g for about 10 minutes. For example, the carbon dot precipitate is repeatedly washed and centrifuged.

For example, the carbon dot precipitate is dried to obtain the carbon dot.

For example, the carbon dot is dried at about dried at about 70° C. to about 100° C. For example, the carbon dot precipitate is dried at about 80° C. to about 90° C. For example, the carbon dot precipitate is dried for about 6 hours to about 48 hours. For example, the carbon dot precipitate is dried for about 24 hours. For example, the carbon dot precipitate is oven-dried.

In some embodiments, the carbon dot is deprotonated. For example, the carbon dot is deprotonated with sodium hydroxide (NaOH), potassium hydroxide (KOH), ammonium hydroxide (NH₄OH), lithium hydroxide (LiOH), calcium hydroxide (Ca(OH)₂) or barium hydroxide (Ba(OH)₂). For example, the carbon dot is deprotonated with sodium hydroxide.

For example, the glycine-citric acid carbon dot has an average particle size of 8 to 14 nm and/or a Gaussian particle size distribution ranging from 6 to 16 nm.

For example, the glycine-citric acid carbon dot has a UV-Vis absorbance spectrum showing two absorbance bands from 190-210 nm and 290-350 nm.

For example, the glycine-citric acid carbon dot exhibits a maximum fluorescence emission at 400 nm when excited at 300 nm.

For example, the glycine-citric acid carbon dot is characterized by at least one of the following FT-IR spectroscopy vibrations:
a stretching vibration at 1658 cm⁻¹,
symmetric and asymmetric stretching vibrations present at 1387 cm⁻¹ and 1582 cm⁻¹, and
stretching vibration at 1302 cm⁻¹.

For example, the glycine-citric acid carbon dot is characterized by the following FT-IR spectroscopy vibrations:
a stretching vibration at 1658 cm⁻¹,
symmetric and asymmetric stretching vibrations present at 1387 cm⁻¹ and 1582 cm⁻¹, and
stretching vibration at 1302 cm⁻¹.

For example, the glycine-citric acid carbon dot is characterized by the following FT-IR spectroscopy vibrations:
a stretching vibration at 1658 cm⁻¹ attributed to N—H moieties of glycines;
symmetric and asymmetric stretching vibrations present at 1387 cm⁻¹ and 1582 cm⁻¹ attributed to deprotonated COO— groups; and
stretching vibration at 1302 cm⁻¹ attributed to carboxylate functional groups.

For example, the glycine-citric acid carbon dot comprises at least one of:
a stretching vibration at 1658 cm⁻¹;
symmetric and asymmetric stretching vibrations present at 1387 cm⁻¹ and 1582 cm⁻¹; and
stretching vibration at 1302 cm⁻¹, and according to FT-IR spectroscopy.

For example, the glycine-citric acid carbon dot comprises at least one N—H group and/or at least one COO⁻ group at a surface thereon.

For example, the glycine-citric acid carbon dot comprises glycine-citric acid carbon dot comprises at least one

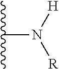

group or at least one

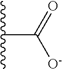 [SMILES: *C(=O)[O-]]

group at a surface thereon,

wherein R is a group chosen from H or a C1-C20 alkyl group.

For example, the glycine-citric acid carbon dot comprises glycine-citric acid carbon dot comprises at least one carboxylate functional group at a surface thereon.

For example, the glycine-citric acid carbon dot comprises glycine-citric acid carbon is comprised of about 10 wt % to 14 wt % of sodium.

For example, the glycine-citric acid carbon dot comprises binding energies at 1071.64, 532.39, 498.03, 399.63, and 286.94 eV, according to XPS survey scan.

For example, the glycine-citric acid carbon dot comprises binding energies at 1072, 532, 498, 400, and 287 eV, according to XPS survey scan.

For example, the glycine-citric acid carbon dot comprises five binding energies at 1071.64, 532.39, 498.03, 399.63, and 286.94 eV corresponding to sodium (Na1s), oxygen (01s), sodium (Na KLL), nitrogen, (Nis) and carbon (C1s), respectively, according to XPS survey scan.

For example, the glycine-citric acid carbon dot comprises five binding energies at 1072, 532, 498, 400, and 287 eV corresponding to sodium (Na1s), oxygen (01s), sodium (Na KLL), nitrogen, (N1s) and carbon (C1s), respectively, according to XPS survey scan.

For example, upon deconvolution of the 01s peak of the glycine-citric acid carbon dot, three binding energies are observed at 530.81, 531.87 and 535.29 eV.

For example, upon deconvolution of the 01s peak of the glycine-citric acid carbon dot, three binding energies are observed at 530.81, 531.87 and 535.29 eV, attributed to the presence of C═O, COOH functional groups and residual sodium.

For example, upon deconvolution of the N1s peak of the glycine-citric acid carbon dot, three binding energies are observed at 398.19, 399.62, and 400.54 eV.

For example, upon deconvolution of the N1s peak of the glycine-citric acid carbon dot, three binding energies are observed at 398.19, 399.62, and 400.54 eV and are attributed to pyridinic nitrogen, benzenoid amine and graphitic nitrogen, respectively.

For example, upon deconvolution of the C1s peak of the glycine-citric acid carbon dot, four binding energies are shown at 284.66, 285.52, 286.10 and 287.79 eV.

For example, upon deconvolution of the C1s peak of the glycine-citric acid carbon dot, four binding energies are observed at 284.66, 285.52, 286.10 and 287.79 eV and are attributed to C═O, C—C, C—O/C—N and C═O functional groups, respectively.

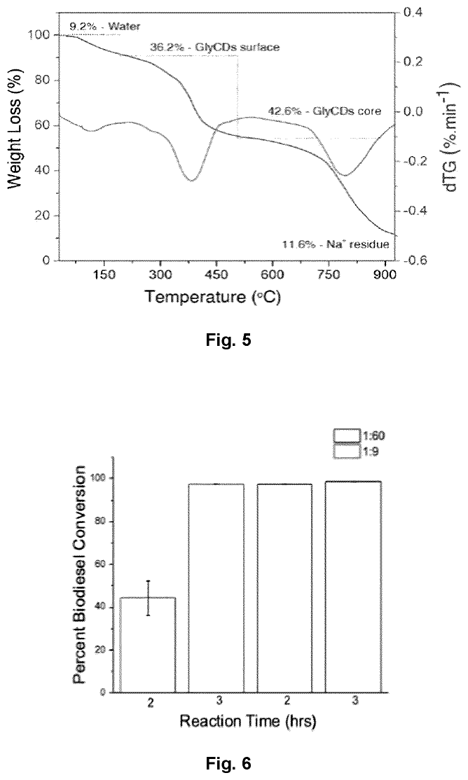

For example, the glycine-citric acid carbon dot has a thermogravimetric analysis curve corresponding substantially to the thermogravimetric analysis curve as shown in FIG. 5.

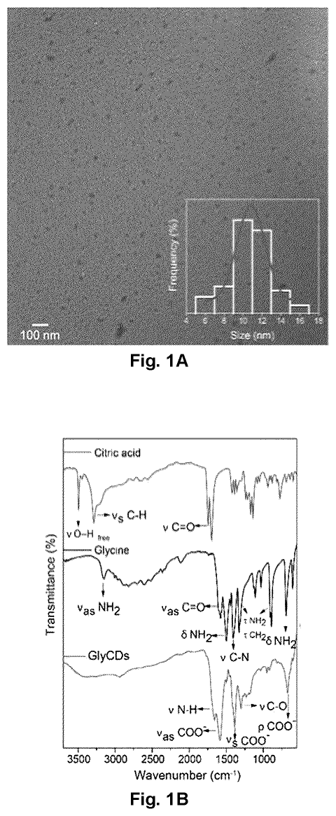

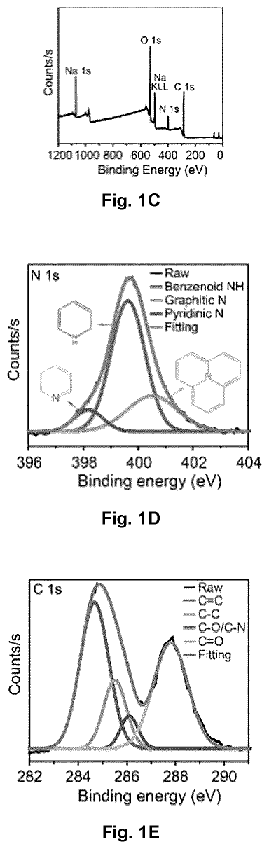

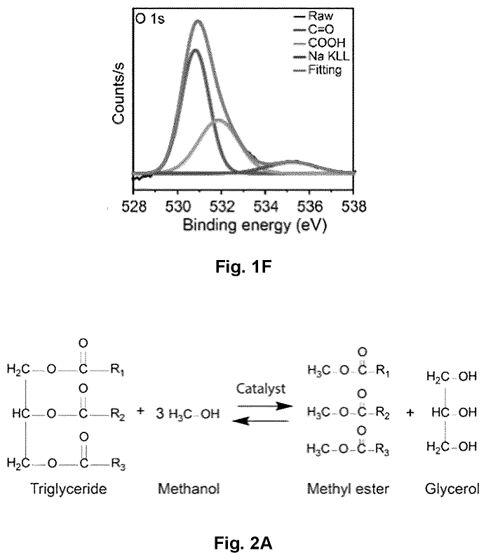

For example, the glycine-citric acid carbon dot has an infrared spectrum corresponding substantially to the representative infrared spectrum as shown in FIG. 1b).

For example, the glycine-citric acid carbon dot described herein is used to catalyze a transesterification reaction.

For example, the glycine-citric acid carbon dot described herein is used to carry out a transesterification reaction of an oil into biofuel.

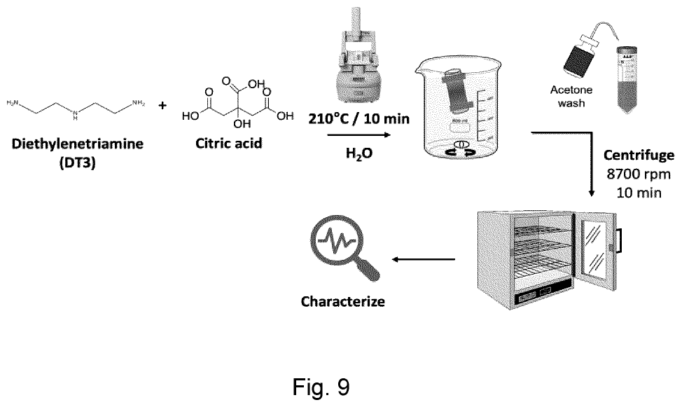

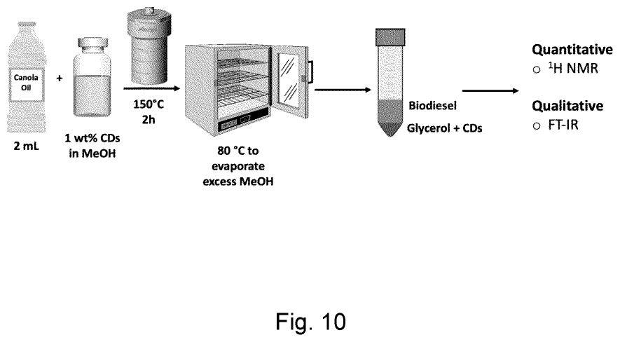

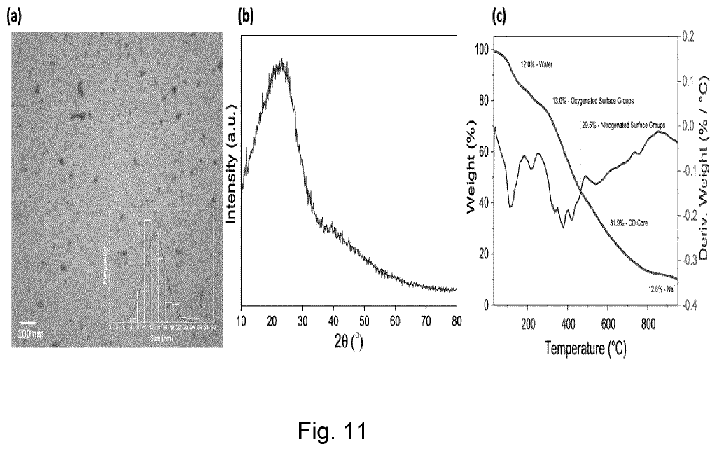

For example, the amine-passivated citric acid carbon dot has a characteristic shown in at least one of FIG. 9(a), FIG. 9(b), FIG. 9(c), FIG. 10(a), FIG. 10(b), FIG. 10(c), FIG. 10(d), FIG. 11(a), and FIG. 11(b). For example, the amine-passivated citric acid carbon dot is as described in the present description and FIGS. 9 to 11.

For example, the amine-passivated citric acid carbon dot has an average particle size distribution of about 5 nm to about 25 nm. For example, the amine-passivated citric acid carbon dot has an average particle size distribution of about 10 nm to about 20 nm. For example, the amine-passivated citric acid carbon dot has an average particle size distribution of about 10 nm to about 16 nm. For example, the amine-passivated citric acid carbon dot has an average particle size distribution up to about 17 nm. For example, the amine-passivated citric acid carbon dot has an average particle size distribution of less than about 40 nm.

For example, the amine-passivated citric acid carbon dot has a Gaussian particle size distribution of about 6 nm to about 26 nm.

For example, the amine-passivated citric acid carbon dot has a UV-vis absorbance spectrum showing two absorbance bands from about 220 nm to about 250 nm and from about 335 nm to about 365 nm.

For example, the amine-passivated citric acid carbon dot has a UV-vis absorbance spectrum showing two absorbance bands at about 240 nm and at about 355 nm.

For example, the amine-passivated citric acid carbon dot has a maximum fluorescence emission in the blue fluorescence region when excited at about 365 nm.

For example, the amine-passivated citric acid carbon dot has a maximum fluorescence emission at about 445 nm, when excited at about 355 nm.

For example, the amine-passivated citric acid carbon dot is characterised by at least one of the following FT-IR spectroscopy vibrations:

a stretching vibration at about 3380 cm⁻¹ and/or at about 3255 cm⁻¹;

a stretching vibration at about 1695 cm⁻¹ and/or at about 1645 cm⁻¹;

a stretching vibration at about 1540 cm⁻¹; and

a stretching vibration at about 1360 cm⁻¹.

For example, the amine-passivated citric acid carbon dot is characterised by the following FT-IR spectroscopy vibrations:

stretching vibrations at about 3380 cm⁻¹ and at about 3255 cm⁻¹;

stretching vibrations at about 1695 cm⁻¹ and at about 1645 cm⁻¹;

a stretching vibration at about 1540 cm⁻¹; and

a stretching vibration at about 1360 cm⁻¹.

For example, the amine-passivated citric acid carbon dot is characterised by the following FT-IR spectroscopy vibrations:

stretching vibrations at about 3380 cm⁻¹ attributed to OH stretching and at about 3255 cm⁻¹ attributed to NH stretching;

stretching vibrations at about 1695 cm⁻¹ attributed to C═O stretching of carboxylic acid or carboxylate and at about 1645 cm⁻¹ attributed to C═O of amide;

a stretching vibration at about 1540 cm⁻¹ attributed to C═C of polyaromatic rings; and

a stretching vibration at about 1360 cm⁻¹ attributed to symmetric carboxylate stretching.

For example, the amine-passivated citric acid carbon dot comprises on a surface thereon at least one function group selected from OH, NH, carboxylic acid, carboxylate, amide, polyaromatic rings and combinations thereof.

For example, the amine-passivated citric acid carbon dot comprises about 10% w/w to about 15% w/w of sodium.

For example, the transesterification is carried out by using carbon dots as heterogeneous catalyst. For example, the transesterification is carried out by using carbon dots herein described as heterogeneous catalyst.

For example, the carbon dot herein disclosed is used as heterogeneous catalyst to catalyze a transesterification reaction.

Methods for carrying out a transesterification reaction and for preparing a biofuel are disclosed herein in other aspects.

For example, the carbon dot catalyst is a heterogeneous carbon dot catalyst.

For example, the oil is a vegetable oil, a waste oil, a grease trap oil or mixtures thereof. For example, the oil is canola oil.

For example, the transesterification reaction is carried out at a temperature of about 50° C. to about 250° C. For example, the transesterification reaction is carried out at a temperature of about 100° C. to about 200° C. For example, the transesterification reaction is carried out at a temperature of about 125° C. to about 175° C. For example, the transesterification reaction is carried out at a temperature of about 140° C. to about 160° C. For example, the transesterification reaction is carried out at a temperature of about 150° C.

For example, the transesterification reaction is carried out with a catalyst loading of about 0.1 to about 40 wt %. For example, the transesterification reaction is carried out with a catalyst loading of about 0.2 to about 20 wt %. For example, the transesterification reaction is carried out with a catalyst loading of about 0.5 to about 10 wt %. For example, the transesterification reaction is carried out with a catalyst loading of about 0.8 to about 1.5 wt %. For example, the transesterification reaction is carried out with a catalyst loading of about 1.0 wt %.

For example, the transesterification reaction is carried out in an oil:alcohol ratio of about 1:40 to about 1:80. For example, the transesterification reaction is carried out in an oil:alcohol ratio of about 1:50 to about 1:70. For example, the transesterification reaction is carried out in an oil:alcohol ratio of about 1:60.

For example, the transesterification is carried out in an oil:alcohol ratio of about 1:15 to about 1:35. For example, the transesterification is carried out in an oil:alcohol ratio of about 1:25 to about 1:30. For example, the transesterification is carried out in an oil:alcohol ratio of about 1:27.

For example, the alcohol is methanol.

For example, the transesterification reaction is carried out in the presence of glycine-citric acid carbon dots (GlyCDs), amine-passivated citric acid carbon dots (amine-passivated CDs), and combinations thereof.

For example, the biofuel is biodiesel.

For example, the carbon dot is a carbon dot as defined herein. For example, the carbon catalyst comprises a carbon dot as defined herein.

### EXAMPLES

These examples are not to be construed as limiting the scope of the present disclosure in any way.

### Example 1 Glycine Citric Acid Carbon Dots

### Materials and Reagents

Glycine (≥99%) and citric acid (≥99.5%) were purchased from Sigma-Aldrich. Sodium hydroxide solution and methanol were purchased from Fisher Scientific. Food grade canola oil was purchased from a grocery store. The Cellulose dialysis membrane (molecular weight cut-off=3.5-5.0 kDa) was purchased from Spectrum labs. All the chemicals were used without further purification.

### Synthesis of Glycine-Citric Acid Carbon Dots (GlyCDs)

GlyCDs were synthesized via a one-step hydrothermal reaction. Briefly, 8.0 mL of sodium hydroxide solution (5 mM) was added to 2.4 g of glycine and gently stirred for 5 min. This was followed by the addition of 10.6 g of citric acid and 100.0 mL of deionized water to the reaction vessel and was stirred until it was clear and homogenous. The solution was then transferred to the hydrothermal reactor and heated at 210° C. under stirring (550 rpm) for 4 hours. Fluorophores are possible reaction intermediates that can be formed during carbon dot synthesis. The dots may be purified using centrifugation, dialysis and organic washes to remove any potential impurities or fluorophores. Specifically, the reaction was left to cool to room temperature and the CD solution was dialyzed for 4 days against water to remove unreacted precursors and fluorophores. Next, the sample was washed multiple times with acetone (1:10 v/v) to remove any remaining impurities. After each wash, the precipitate was collected by centrifugation at 10000×g for 10 minutes. The resultant dark brown precipitate was dried in an oven at 85° C. for 48 hours.

### Transesterification of Canola Oil to Biodiesel Using GlyCDs Catalyst

A known amount of GlyCDs was added to methanol until a homogenous dispersion was obtained. It was then added to 2.0 mL of canola oil and the mixture was transferred to a 25.0 mL hydrothermal reactor. The reaction was carried out at various conditions as summarized in Table 1. For instance, for canola oil:MeOH molar ratio of 1:60, a total of 12.5 mL of methanol was used. Following reaction completion, the reactor was cooled to room temperature and the multiphase biodiesel solution was transferred to a vial and placed in a 65° C. oven to allow the residual methanol to evaporate. The top phase (i.e. the biodiesel) was separated by centrifugation at 10,000×g for 5 min from the lower GlyCDs-glycerol layer and was further used for analysis.

TABLE 1
Reaction parameters varied in order to determine optimal
conditions for the transesterification of canola oil using
glycine-citric acid CDs.

| GlyCD | | Reaction | Canola |
|---|---|---|---|
| Loading | Temperature | time | Oil:MeOH |
| (wt %) | (° C.) | (hrs) | (molar ratio) |
| 0.1 | 150 | 3 | 1:60 |
| 0.25 | 150 | 3 | 1:60 |
| 1.0 | 65 | 3 | 1:60 |
| 1.0 | 100 | 3 | 1:60 |
| 1.0 | 125 | 3 | 1:60 |
| 1.0 | 150 | 3 | 1:18 |
| 1.0 | 150 | 3 | 1:60 |
| 1.0 | 150 | 2 | 1:18 |
| 1.0 | 150 | 2 | 1:60 |
| 1.0 | 150 | 1 | 1:60 |
| 10 | 150 | 3 | 1:60 |
| 20 | 150 | 3 | 1:60 |

### Characterization Techniques

Transmission Electronic Microscopy (TEM). GlyCDs were dispersed in MeOH at a concentration of 10.0 mg·mL⁻¹. TEM grids were prepared by pipetting 10.0 μL of the GlyCD solution on a 300 Mesh Cu (Cu-300HD) coated with a holey/thin carbon film (Grid Tech). The solvent was allowed to evaporate prior to analysis. TEM analysis was carried out using an LVEM5 benchtop electron microscope operating at 5 kV. Size distribution analysis was carried out using Fiji imaging software.

UV-Vis Absorbance Spectroscopy. UV-Vis absorption spectra of aqueous dispersions of the GlyCDs (20.0 μg·mL⁻¹) were acquired in the spectral range of 180-800 nm on a Cary 5 Series UV-Vis-NIR Spectrophotometer (Agilent Technologies) using a 1 cm quartz cuvette. A 5.0 nm bandwidth and wavelength changeover at 450 nm were used for analysis. The data was analyzed using the Cary Eclipse software.

Fluorescence Spectroscopy. Fluorescence spectra were collected using a Cary Eclipse fluorescence spectrophotometer (Agilent Technologies). Spectra were acquired in a 1 cm quartz cuvette at λex=300 nm (5 nm intervals). The excitation and emission slits were set to a width of 5 nm with a PMT voltage at 600 V. Data was processed using Cary Eclipse software.

Fourier-Transform Infrared Spectroscopy (FT-IR). FT-I R spectra were collected using a Thermo Scientific Nicolet iS5 equipped with an iD5 ATR accessory. Spectra were collected using 32 scans with a resolution of 0.4 cm-1, a gain of 1, an optical velocity 0.4747 and an aperture setting of 100. Data was processed using Omnic 9 software.

X-Ray Photoelectron Spectroscopy (XPS). XPS analysis of GlyCDs was acquired using a Thermo Scientific K-Alpha x-ray photoelectron spectrometer. Each analysis was carried out on three different points of the sample, in triplicate, with 10 runs for each scan. The averages were plotted for both the survey and high-resolution scans.

Thermogravimetric Analysis (TGA). Thermogravimetric analysis was carried out using a TGA Q500 analyzer from TA instruments. Samples were heated from 25 to 900° C. at a heating rate of 5° C.·min−1 under an N2 atmosphere with a flow rate of 50 mL·min−1.

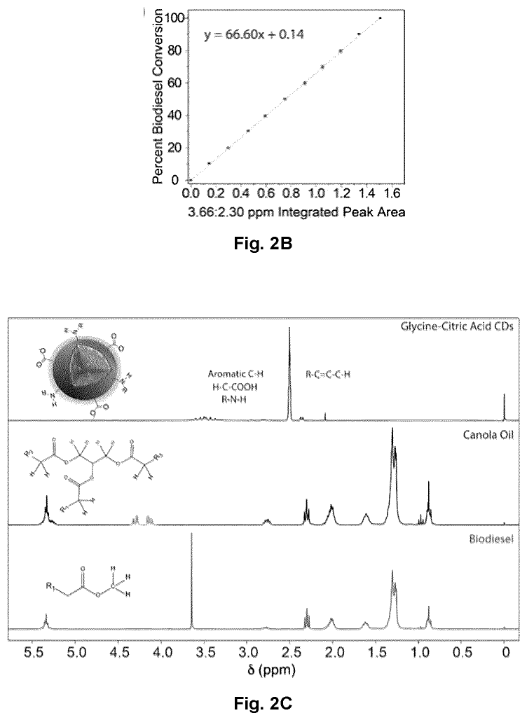

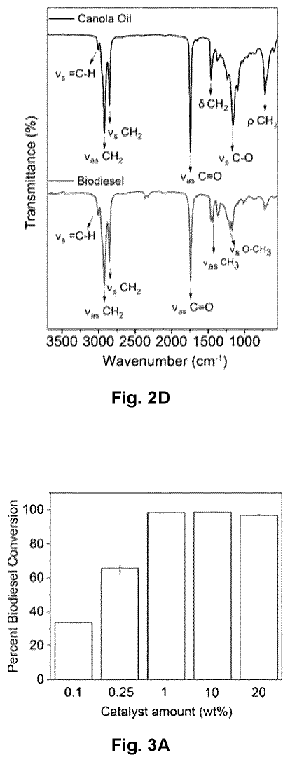

Characterization of Biodiesel via Proton Nuclear Magnetic Resonance (¹H NMR). Quantitation and composition of biodiesel was determined using a Bruker Fourier Ultrashield™ operating at 300 MHz. Sample analysis was carried out using the Mestrelab Mnova software. All samples were prepared in CDCl3 for analysis. A calibration curve was obtained by preparing 10 known sample mixtures of methyl oleate and canola oil from 0% to 100% methyl oleate. The ratio between the integrated peaks at 3.66 ppm and 2.30 ppm were calculated and plotted against the known percentage of methyl oleate present in the sample. This method was also used to quantitate the conversion of biodiesel obtained through the GlyCD catalysed transesterification reactions. In this case, the ratio of the integrated peaks (3.66 ppm/2.30 ppm) was calculated and the percent conversion value was obtained from the linear regression equation of the calibration curve (FIG. 2b).

### Results and Discussion

### Synthesis and Physical Characterization of the Carbon Dots

It is herein reported the synthesis of CDs from glycine and citric acid precursors via a one-step hydrothermal reaction at 210° C. for a period of 4 hours after which the reaction solution was dialyzed and subjected to organic washes to ensure removal of unreacted precursors and intermediates. The Hydrothermal route offers several advantages namely as it allows for the use of larger sample volumes and is relatively inexpensive.³⁴⁻³⁶

Transmission Electron Microscopy (TEM) analysis shows a monodispersed sample of quasi-spherical dots with an average size of 10.9±2.8 nm (FIG. 1a). Particle size distribution of over 100 dots shows a Gaussian size distribution (FIG. 1a inset) ranging from ˜6-16 nm. The optical properties of the GlyCDs were characterized by UV-Vis and fluorescence (PL) spectroscopy (FIG. 4). The UV-Vis absorbance spectrum shows two absorbance bands from 190-210 nm and 290-350 nm. The first band can be assigned to the π→π* transition attributed to alkenes and aromatic sp² domains, while the second band can be assigned to the n→π* transitions which are attributed to functional groups, such as carboxyl and amides.³⁷⁻³⁹ The GlyCDs exhibit a maximum fluorescence emission at 400 nm when excited at 300 nm. While the optical properties do not play a role in the transesterification reaction, they are used to confirm along with the physical characterization techniques (see below), their formation.

Surface properties of heterogeneous catalysts are important in regulating catalytic efficiency since the reaction is occurring on the surface of the catalyst.⁴⁰ FT-IR spectroscopy was used to characterize the surface of the GlyCDs (FIG. 1b) and are compared to the glycine and citric acid precursors. The glycine precursor presented vibrations associated with its amino acid structure, presenting NH₂ and C═O functionalities at 3154 cm⁻¹ and 1587 cm⁻¹, respectively, as previously reported in the literature.⁴¹ The citric acid precursor presents vibrations associated to O—H and C═O functionalities at 3491 cm⁻¹ and 1730 cm⁻¹, respectively, as previously reported in the literature.⁴² GlyCDs presented characteristic vibrations associated to the precursors used during synthesis. A stretching vibration at 1658 cm⁻¹ can be attributed to the N—H moieties of the glycine amino groups. Symmetric and asymmetric stretching vibrations present at 1387 cm⁻¹ and 1582 cm⁻¹, respectively, are attributed to deprotonated COO⁻ groups present on the CD surface. Deprotonation of the carboxylic groups occurs through the use of NaOH during synthesis and is necessary to endow alkaline characteristics to the surface of the GlyCDs. A stretching vibration at 1302 cm⁻¹ is attributed to C—O moieties resulting from the carboxylate functional groups.

The FT-IR results are complementary to the analysis obtained by XPS (FIG. 1c), FIG. 1d), FIG. 1e) or FIG. 1f). The XPS survey scan reveals five binding energies at 1071.64, 532.39, 498.03, 399.63, and 286.94 eV corresponding to sodium (Na1s), oxygen (O1s), sodium (Na KLL), nitrogen, (Nis) and carbon (C1s), respectively (FIG. 1c)). High resolution XPS (HR-XPS) analysis of the binding energies present in the survey spectrum and subsequent deconvolution of the observed peaks was used to determine the chemical states at the surface (and core) of the CDs. Upon deconvolution of the O1s peak, three binding energies are observed at 530.81, 531.87 and 535.29 eV, attributed to the presence of C═O, COOH functional groups and residual sodium (FIG. 1f)). The residual sodium behaves like counter ions to stabilize the carboxylates present on the CD surface. Deconvolution of the N1s peak shows three binding energies observed at 398.19, 399.62, and 400.54 eV and are attributed to pyridinic nitrogen, benzenoid amine and graphitic nitrogen, respectively (FIG. 1d)). Deconvolution of the C1s peak (at FIG. 1e)) shows four binding energies at 284.66, 285.52, 286.10 and 287.79 eV and are attributed to C═O, C—C, C—O/C—N and C═O functional groups, respectively (FIG. 2d).⁴³

As the transesterification reaction is carried out at temperatures of nearly 150° C., thermogravimetric analysis was carried out to determine the thermal stability of the carbon dots. The TGA thermogram (FIG. 5) shows a three-step decomposition pattern. The initial 9.2% weight loss, at 140° C. can be attributed to the removal of water molecules adsorbed to the surface of the carbon dots. The second weight loss (36.2%) occurring at 385° C. is attributed to the onset of decomposition of the surface moieties. The third weight loss step occurs at 795° C. and accounts for 42.6% of the total weight loss, indicating that the CD core has decomposed. The residual weight left over at 900° C. is due to the Na⁺ ions present on the surface of the carbon dots. This is in accordance with the XPS data that shows the presence of sodium making up ˜12% of the total CD composition.

### Characterization of Biodiesel

The transesterification of canola oil is categorized by the breakdown of the triglyceride chain into a monoglyceride units with a methyl ester terminal (FIG. 2a). Conventional methods for characterization and quantification of the methyl ester yield in biodiesel include high-performance liquid chromatography and gas-liquid chromatography; however, apart from requiring standard calibration curves, the often require derivatization and other sample treatment procedures.⁴⁴⁻⁴⁶ In contrast, ¹H NMR can be used to accurately and easily determine biodiesel conversion by integrating the chemical shift at 3.66 ppm pertaining to the methoxy hydrogens present in biodiesel and the chemical shift at 2.30 ppm ascribed to the methylene hydrogens adjacent to the carbonyl of the triglyceride. This approach was used to quantitate the biodiesel conversion based on a calibration curve of known amounts of methyl ester and canola oil (FIG. 2b).⁴⁷ We observe two significant changes to the ¹H NMR spectrum upon conversion of canola oil to biodiesel (FIG. 2c). When the transesterification reaction has successfully gone to completion (biodiesel yield ˜100%), one doublet of doublets at ˜4.30 ppm disappears. This indicates the conversion of the triglyceride backbone in canola oil to form methyl esters in biodiesel. The formation of a singlet at 3.66 ppm attributed to the methoxy protons of the methyl esters as biodiesel is formed. A comparison of the integrated area (and its intensity) under the methoxy singlet relative to other peaks in the spectrum, is indicative of how much canola oil has been converted to biodiesel during the transesterification reaction. ¹H NMR analysis was also performed on the GlyCDs to ensure that there was no singlet present at 3.66 ppm that could have been mistaken as the methyl ester signal in biodiesel, had there been residual GlyCDs present in the reacted oil.

FT-IR can also be used as a qualitative technique to monitor the conversion of canola oil to biodiesel. A significant change is noted at 1143 and 1196 cm⁻¹ (FIG. 2d) where the former arises due to the —CH₃ asymmetric stretching of the methoxy methyl group and the latter to the symmetric O—CH₃ stretching.⁴⁸

### GlyCD Catalyzed Transesterification of Canola Oil

All transesterification reactions were carried out in a 25 mL hydrothermal reactor. In order to optimize the reaction conditions, we investigated several parameters including catalyst loading, canola oil:methanol ratio, reaction time and temperature (Table 1).

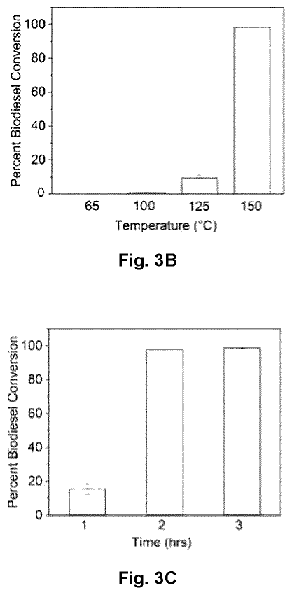

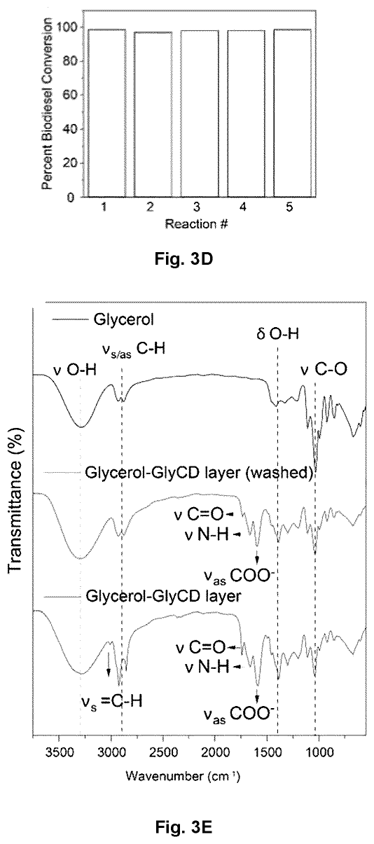

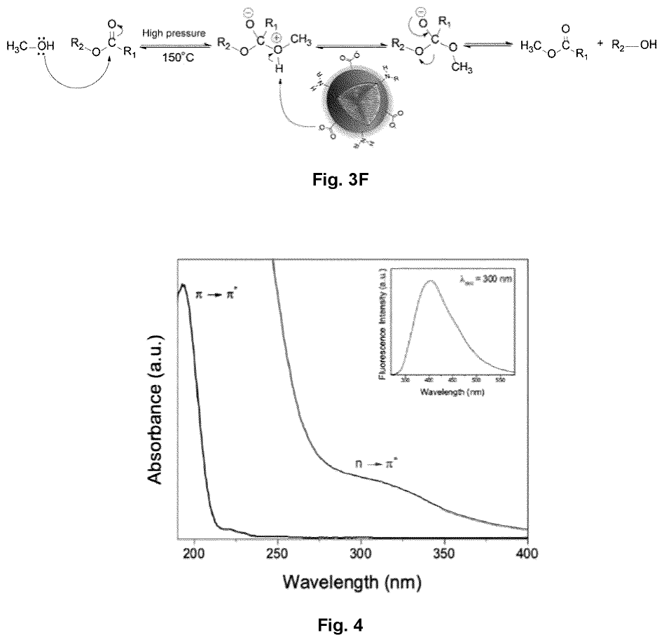

The amount of conventional homogenous (e.g. KOH) catalyst required to catalyze the transesterification of canola oil to biodiesel under conventional heating has been reported to be ˜1 wt % of the oil precursor used.^{11,49} However, previous literature reports have shown that higher amounts are required when using heterogeneous catalysts typically ranging from 3-15%^{7,16} The transesterification reactions were carried out varying GlyCD catalyst in the range of 0.1-20 wt % (FIG. 3a). In order to isolate the effect of catalyst loading, all other parameters were kept constant at 150° C. and 3-hour reaction time with an oil:methanol ratio of 1:60. It is interesting to notice that even at small percentages of catalyst, such as 0.1%, a significant conversion of 30% was obtained. It was found that at 1.0% of GlyCDs, the conversion from canola oil to biodiesel reaches a plateau, with a yield of 98.3±0.2%. For that reason, this amount of catalyst was kept constant for the subsequent optimization steps.

As for any catalyzed reaction, temperature has a large impact on the reaction rate.⁵⁰ Conventionally heated reactions using homogenous basic catalysts such as NaOH, are usually carried out at 65° C. Nevertheless, heterogeneous catalysts often require higher temperature to increase their catalytic efficiency. To evaluate the effect of temperature, the transesterification reaction using GlyCDs was carried out at 65° C., 100° C., 125° C. and 150° C. (FIG. 3b). At the highest temperature tested (150° C.), a biodiesel conversion yield of 98.3±0.2% was obtained. The transesterification reaction was found to be significantly lower at temperatures below 150° C. Conversions of 0%, 0.7±0.05% and 9.2±1.9% were obtained at 65° C., 100° C. and 125° C., respectively. Thus, it was deemed that a temperature of about 150° C. desirable for the GlyCDs catalyzed transesterification reaction.

Subsequently, the effect of reaction time on biodiesel conversion was investigated at 1 h, 2 h and 3 h, while keeping all other parameters constant. (FIG. 3c). At the 1 hr time point, a modest conversion of canola oil to biodiesel is observed with a value of 15.6±2.8%; however, increasing the reaction time to 2 hrs significantly influences the conversion reaction with an observed value of 97.6±0.2%.

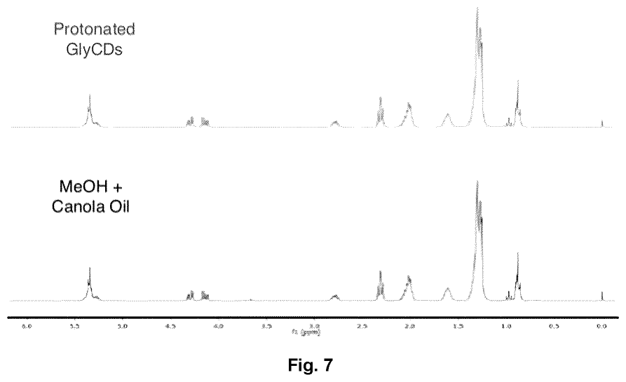

The stoichiometric ratio required for the transesterification reaction is one molecule of oil to three molecules of alcohol. However, this reaction occurs as an equilibrium, and an excess of the alcohol is added to drive the reaction in the forward direction. Normally, when a homogenous catalyst is used to produce biodiesel, an oil to methanol ratio of 1:6 is used. In the case of heterogeneous catalysts, often higher ratios are used reaching 1:90-1:275.^{7,51-53} The ratio of canola oil to methanol was varied from 1:60 to 1:18 to determine the ideal ratiometric volume of excess methanol required for the transesterification reaction. The transesterification reaction was carried out using a reaction time of 2 hrs, and using a 1:18 oil to methanol ratio. These conditions resulted in low biodiesel conversions of 44.2±8.2% (FIG. 6). However, using a 1:9 oil to methanol ratio and reaction time of 3 hrs, biodiesel conversions of 97.6±0.2% was observed, this being the highest biodiesel conversion obtained using optimal conditions. Without wishing to be bound to this theory, it was found that with a 1 wt % catalyst loading at 150° C. with a short reaction time of 3 hrs, and a low oil:methanol ratio (1:18), this system is a promising candidate for the biodiesel production in comparison to already-reported heterogeneously catalyzed systems. In order to ascertain the effects of the CD catalysts, control reactions were carried out to ensure that the efficient biodiesel conversion was not stemming from other sources (FIG. 7). A reaction of solely canola oil and methanol was carried out in the hydrothermal reactor at 150° C. for 3 hours using an oil to methanol ratio of 1:60 and showed that in the absence of the GlyCDs, no conversion to biodiesel can be obtained. Additionally, the use of protonated GlyCDs was investigated, which were synthesized in the exact same way except for the addition of sodium hydroxide. The transesterification reaction was carried out using 20 wt % of protonated GlyCDs at 150° C. for 3 hours using an oil to methanol ratio of 1:60. No singlet peak was observed by ¹H NMR at 3.66 ppm indicating no conversion to biodiesel had occurred. Indeed, the ¹H NMR spectrum were identical to that of the starting canola oil.

### Reusability and Stability of GlyCD Catalyst

One of the primary benefits of using a heterogeneous catalyst is the possibility of recuperating the catalyst once the reaction has gone to completion and its reuse in subsequent reactions. Once the conditions for the transesterification reactions were optimized, the reusability of the GlyCDs were investigated. Upon completion of each reaction, the mixture of biodiesel oil, glycerol and carbon dots was left in the oven at 70° C. to remove the excess methanol. After centrifuging the mixture, separation of the biodiesel from the glycerol/GlyCD mixture was achieved. The second transesterification reaction is carried out using the same conditions as the first reaction and a biodiesel conversion of 97.1% was obtained. This procedure was repeated for 5 catalytic cycles and what is remarkable is that there appears to be no decrease in biodiesel conversion with each subsequent reaction (FIG. 3d). The separation of the phases using FTIR (FIG. 3e) was confirmed where it is clear that the glycerol phase shows a similar spectrum relative to pure glycerol. Some residual biodiesel is also present in the glycerol-GlyCD layer confirmed by the stretch at 3007 cm⁻¹, which represents the symmetric stretching of the sp² C—H in biodiesel. To further purify the glycerol layer containing the GlyCDs, the layer is dispersed in methanol and subjected to multiple organic washes using hexanes to remove any remaining biodiesel. FIG. 3e shows the FT-IR analysis once the glycerol-GlyCD layer was washed with hexanes. Characteristic stretches of glycerol at 2935 cm⁻¹ and 2879 cm⁻¹, pertaining to the symmetric and asymmetric C—H stretching, can be seen, however the sp² C—H stretch found in biodiesel is not present, confirming the removal of residual biodiesel. The results show sustained catalytic efficiency of the GlyCDs while maintaining biodiesel conversions above 96.7%.

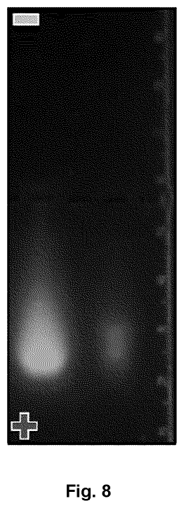

It is believed that the catalytic mechanism works in a similar way to homogenous base catalysed transesterification reaction. In the conventional reaction the deprotonation of methanol occurs due to the use of a strong base (e.g. KOH, NaOH), followed by the methoxide attack on the carbonyl of the triglyceride. It has been reported that some organic compounds such as amino-containing molecules can act as non-ionic bases and catalyze the transesterification reaction.⁵⁶ In this case, the amino group deprotonates the methanol, and the methoxide formed attacks the ester.⁵⁷ The transesterification mechanism can also occur with weak bases, such as carboxylates.⁵⁸ However, higher pressures and reaction temperatures are needed to overcome the energy barrier to allow the reaction to move forward. This validates our findings where temperatures of ˜150° C. in the hydrothermal reactor were desirable in order to ensure biodiesel conversion. It was confirmed by gel electrophoresis that the GlyCDs are in fact negatively charged (FIG. 8). As both amino and carboxylates groups are present on the surface of GlyCDs, both mechanisms are likely to occur. However, the carboxylates are believed to play a major role in the transesterification reaction mechanism. Therefore, the proposed mechanism for the GlyCDs-catalyzed reaction (FIG. 3f) starts with the methanol attack on the ester, forming a tetrahedral intermediate zwitterion, which collapses, and a proton transfer by the carboxylates on the CD surface will occur and shift the reaction equilibrium. Subsequently, the reaction follows the normal transesterification mechanism, forming glycerol as a byproduct. ⁵⁷ We also conducted a control reaction where protonated GlyCDs were used for the transesterification reaction and it was found that a negligible biodiesel conversion was obtained, which can be attributed to the amino groups on the CD surface that may play a small role in the catalyzed reaction.

### Conclusions

The catalytic efficiency of GlyCDs was investigated for the hydrothermal transesterification of canola oil to produce biodiesel composed of fatty acid methyl esters. Multiple reaction parameters were varied in order to determine the optimum conditions to produce the highest biodiesel conversion using the minimum amount of energy and catalyst. Without wishing to be bound by such a theory, the optimized reaction parameters were determined as about 1 wt % GlyCDs catalyst, reacted with about a 1:9 ratio of canola oil to methanol at a temperature of about 150° C. for a duration of about 3 hours. The transesterification of canola oil with GlyCDs under the optimized conditions resulted in a high biodiesel conversion of 97.6±0.2%. The stability and reusability of the GlyCDs was investigated and it was found that they maintain biodiesel conversions above 97% for at least five cycles, however it is believed that catalytic efficiency would be maintained for multiple subsequent transesterification reactions. While other reported heterogeneous catalysts also offer the advantages of catalyst recovery and reusability, several drawbacks are still present such as metal leeching as is the case for metal containing heterogeneous catalysts, as well as the use of expensive precursors and multi-step synthesis procedures. Carbon dots offer the advantages of facile one pot syntheses using inexpensive and organic precursors. Using carbon dots as transesterification catalysts can permit biodiesel production to become a sustainable process and allow it to become an alternative renewable energy source that will meet future demands.

### Example 2 Amine-Passivated Citric Acid Carbon Dots

### Materials and Reagents

Citric acid (CA), ethylenediamine (ED2), diethylenetriamine (DT3), triethylenetetramine (TT4), tetraethylenepentamine (TP5), pentaethylenehexamine (PH6) and acetone were purchased from Sigma-Aldrich. Sodium hydroxide and methanol were purchased from Fisher Scientific. Food grade canola oil was purchased from a grocery store. Cellulose dialysis membrane (3.5-5.0 kDa molecular weight cut-off) was purchased from Spectrum Laboratories. All reagents were used without further modification or purification.

### Synthesis of Amine-Passivated Citric Acid Carbon Dots

CDs were synthesized via a one-step microwave-assisted reaction of citric acid and the amine-passivating agent (ED2, DT3, TT4, TP5 or PH6) using a CEM Discover™ SP microwave reactor (FIG. 9). Briefly, 1.5 g (500 mM) of citric acid and 16.0 mL of Milli-Q™ water were mixed in a glass microwave-transparent reaction vial and stirred until the solution was homogenous. This was followed by the addition of the amine-passivating agent (the primary and/or the secondary amine), where the concentration ranged from 125 mM to 750 mM (Table 2). The reaction vessel was heated to 210° C. for 10 min under constant stirring. Upon completion of the reaction and subsequent cooling to room temperature, the crude CD solutions were dialyzed for 4 days against Milli-Q water, changing the water once a day, allowing for the removal of unreacted precursors and polar impurities. Subsequently, the CDs were further purified using 3 acetone organic washes (1:10 v/v), removing any remaining impurities. Following each organic wash, the sample was vortexed for approximately 30 seconds and centrifuged at 10 000×g for 10 minutes. Subsequently, the supernatant was discarded, and the wash repeated. After the third acetone wash, the resulting precipitate was dried in an oven at 85° C. for 24 hours.

TABLE 2
Concentration and type of amine passivating agent

| Concentration of passivating agent | | Type of passivating agent | |
|---|---|---|---|
| concentration | Volume | Passivating | Volume |
| mM | (μL) | Agent | (μL) |
| 125 | 216 | ED2 | 400 |
| 250 | 432 | TT4 | 893 |
| 375 | 648 | TP5 | 1138 |
| 750 | 1296 | PH6 | 1468 |

### Deprotonation of Amine Passivated Citric Acid Carbon Dots

Briefly, 0.1 g of the amine-passivated CDs were dispersed in 10 mL of Milli-Q water and sonicated for 10 minutes. A pH electrode was used to determine the initial pH of the aqueous CD dispersions and 0.01 M NaOH solution was added dropwise to the samples under constant stirring until the pH of the solution was raised by approximately 2 pH values. The samples were further purified using 3 acetone organic washes, following the same procedure employed during the original purification of the synthesized CDs.

### CD-Catalysed Transesterification of Canola Oil to Biodiesel

CD-catalysed transesterification reactions were for example carried out according to FIG. 10. Approximately 0.5-10 wt % of amine-passivated CDs (wt % relative to canola oil) was added to methanol (0.75-6 mL). The solution was sonicated for 5 minutes until the CDs were homogenously dispersed. Subsequently, 2.0 mL of both canola oil and the CD dispersion were simultaneously transferred to a 25 mL solvothermal autoclave reactor. The reaction was heated at 150° C. for 3 hours under constant stirring at 600 rpm. Upon completion of the reaction, the reactor was submerged in ice in order to rapidly cool the reaction. The reaction mixture was then transferred to a vial and heated in an oven at 85° C. for 12 hours to ensure excess methanol evaporation and separation of the glycerol/CDs and biodiesel phases. The biodiesel phase was then centrifuged at 10 000×g for 5 minutes to further separate the layer and the biodiesel phase collected for ¹H NMR analysis.

### Characterization Techniques

Transmission electron microscopy (TEM). TEM images were acquired using a JEM-2100F™ (JEOL Ltd) analytical electron microscope under a 5-kV field emission. CDs were dispersed in methanol at a concentration of 10 mg mL⁻¹ and 10 μL of the CD dispersions were pipetted on 300 Mesh Cu (Cu-300 HD) TEM grids coated with a holey/thin carbon film (Grid Tech). The solvent was allowed to evaporate prior to image acquisition. Image processing and size distribution analysis were carried out using the Fiji™ imaging software.

X-ray powder diffraction (XRD). XRD spectra were acquired using a 2nd Gen D2 Phaser™ X-ray diffractometer (Bruker AXS). A Cu Kα source at a generator power of 30 kV/10 mA, a scan range from 10 to 80° (29) with a step size of 0.11° per 2 s, and a position sensitive detector opening of 4.79° were used during analysis. All data was processed using the Bruker DIFFRAC™ software.

UV-Vis absorbance spectroscopy. UV-visible absorption spectra were acquired using a Cary™ 5 Series UV-Vis-NIR Spectrophotometer (Agilent Technologies). CDs were dispersed in Milli-Q water at a concentration of 75 μg mL⁻¹ and transferred to a 1 cm quartz cuvette. Samples were analysed at a scan range of 200-800 nm. A scan rate of 600 nm min⁻¹, a resolution of 1 nm and a bandwidth of 5 nm were used during absorption spectra acquisition. All data was processed using the Cary Eclipse™ Scan software.

Fluorescence spectroscopy. Fluorescence spectra were acquired using a Cary Eclipse™ Fluorescence Spectrophotometer (Agilent Technologies). CDs were dispersed in Milli-Q water at a concentration of 0.75 mL⁻¹ and transferred to a 1 cm quartz cuvette. Samples were analysed at an Δₑₓ of 350 nm and an λₑₘ scan range of 370-600 nm with excitation and emission slit widths both set to 5 nm. A scan rate of 600 nm min⁻¹, an emission filter of 295-1100 nm and an emission PMT of 600 V were used during the fluorescence spectra acquisition. All data was processed using the Cary Eclipse software.

Fourier-transform infrared spectroscopy (FT-IR). FTIR-ATR spectra were acquired using a Thermo Scientific Nicolet iS5™ equipped with an iD5 ATR accessory. Dried CD samples were analysed on a laminate-diamond crystal window using 32 scans at 0.4 cm⁻¹ resolution. The detector was set to a gain of 1, an optical velocity of 0.4747 cm/s and an aperture setting of 100. All data was processed using the Thermo Scientific Nicolet Omnic 9 software.

Thermogravimetric analysis (TGA). TGA was performed on dried CD samples in Pt pans using a TGA Q500™ analyser (TA Instruments). Samples were analysed under a N₂ atmosphere at a flow rate of 50 mL min⁻¹ and were heated from 25 to 1000° C. at a heating rate of 1° C. min⁻¹. All data was processed using the Thermal Advantage 5.0 software.

Zeta-potential analysis. Zeta-potential measurements were acquired using a Zeta Size™ (Malvern Instruments —Nano Series). CD samples were dispersed in water at a concentration 5 mg mL⁻¹ and were analysed in triplicates of 20 runs each. The average result of the triplicate was used.

Characterization of biodiesel via proton nuclear magnetic resonance (1H NMR). ¹H NMR spectra were acquired using a Bruker Fourier Ultrashield™ with an operating frequency of 300 MHz. Biodiesel samples were diluted in CDCl3 prior to analysis. Biodiesel conversions were determined based on a calibration curve previously obtained by Macina et al.⁵⁹ The ratio between the integrations of peaks at 3.66 and 2.30 ppm was used to determine the percent conversion by inputting the value into the linear regression equation.

### Results and Discussion

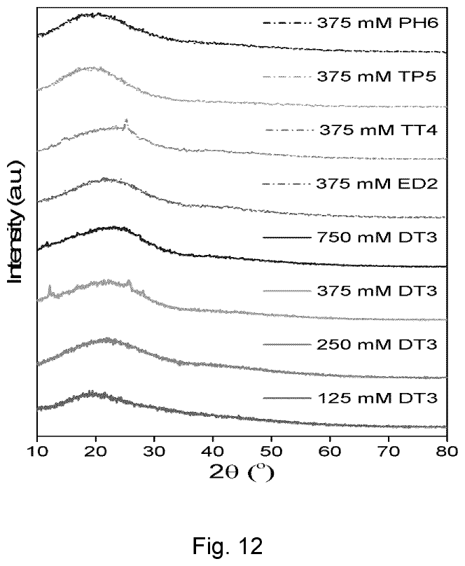

Physical and optical properties of amine-passivated CDs. The CDs' morphology and size distribution were investigated by TEM. FIG. 11a shows fairly monodispersed, quasi-spherical dots with average sizes of 13.2±3.1 nm for the deprotonated 750 mM DT3-CDs and little signs of agglomeration were displayed. Statistical analysis revealed a Gaussian size distribution ranging from 6 to 26 nm (FIG. 11a inset). Since the deprotonation process only interferes with the functional groups on the surface of the CDs, the size distribution remains relatively consistent with their protonated counterparts. XRD analysis (FIG. 12) confirmed that the protonated CDs were amorphous in nature. This was evidenced by an amorphous halo observed in the diffraction pattern along with the absence of any crystalline reflections. XRD analysis (FIG. 11) was also carried out for deprotonated 750 mM DT3-CDs to confirm the retention of their amorphous nature upon deprotonation with sodium hydroxide. Once again, the representative diffractogram shows an amorphous halo and no crystalline reflections, suggesting deprotonation had no impact on their physical structure.

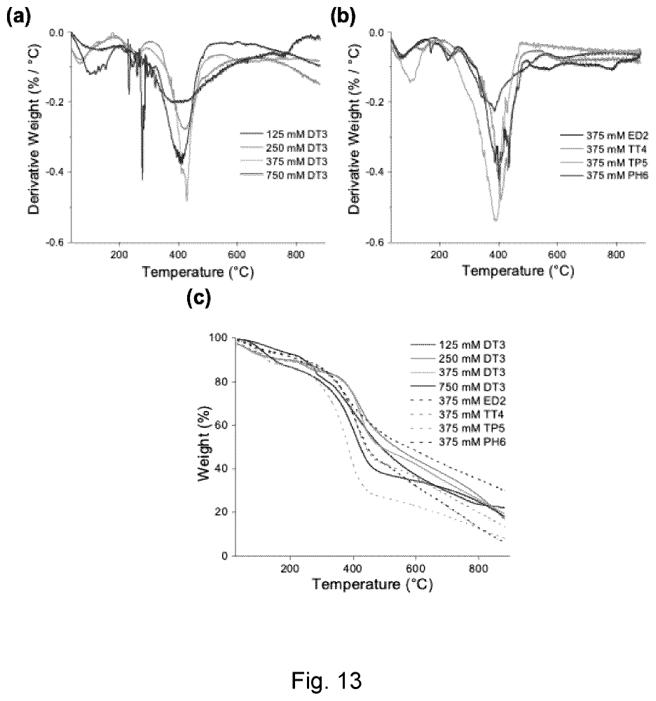

TGA was carried out on the deprotonated CDs to determine their thermal stability, noting that a high thermal stability is a prerequisite for the CD catalysts due to reaction temperatures reaching 150° C. A representative TGA thermogram (FIG. 11c) of the deprotonated 750 mM DT3-CDs shows a four-step decomposition weight loss profile. The initial 12.0% weight loss occurs at 150° C. and is attributed to the evaporation of water adsorbed to the surface of the CD.⁶⁰ The second weight loss of 13% occurring at 310° C. is attributed to the decomposition of oxygenated surface groups whereas the third weight loss of 29.5% occurring at 465° C. is attributed to the decomposition of nitrogenated surface groups.^{61, 62} The final weight loss (31.9%) occurs at 815° C. and is related to the decomposition of the sp² carbon core.⁶³ Finally, the 12.6% residual weight remaining at 900° C. is caused by Na⁺ ions coordinated to the surface of the CD upon deprotonation with NaOH, also observed using XPS where we note the corresponding Na KLL binding energy.⁵⁹ Differential thermogravimetry curves and TGA thermograms (FIG. 13) show similar four-step decomposition profiles and weight-loss steps for all other amine-passivated CDs. The second weight loss attributed to the decomposition of oxygenated surface groups is supported by a pronounced weight loss observed for the 125 mM DT3-CDs (FIG. 13a,c), which contain the highest concentration of oxygenated surface groups relative to the other DT3-CDs (Table 3). The third weight loss, attributed to the decomposition of nitrogenated surface groups, is supported by the large weight losses (20-30%) for the 375 mM DT3-, 750 mM DT3-, 375 mM TT4-, 375 mM TP5- and 375 mM PH6-CDs (FIG. 13b,c). These dots comprise higher concentrations of nitrogenated surface groups relative to the other amine-passivated CDs (Table 3). Thermogravimetric analysis confirms a high thermal stability for all synthesized amine-passivated CDs and the CD surface moieties, which are key catalytic factors.

TABLE 3
Elemental analysis of protonated and deprotonated
amine-passivated citric acid carbon dots

| Amine- | | | | | | | | | | | | |
|---|---|---|---|---|---|---|---|---|---|---|---|---|
| Passivated | Protonated | | | | | | Deprotonated | | | | | |
| CDs | N (%) | C (%) | H (%) | O (%) | N/C | O/C | N (%) | C (%) | H (%) | O (%) | N/C | O/C |
| 125 mM DT3 | 12.76 | 59.97 | 5.35 | 21.92 | 0.21 | 0.37 | 11.33 | 58.66 | 6.51 | 23.50 | 0.19 | 0.40 |
| 250 mM DT3 | 15.24 | 61.26 | 5.95 | 17.55 | 0.25 | 0.29 | 14.82 | 57.49 | 6.06 | 21.63 | 0.26 | 0.38 |
| 375 mM DT3 | 17.48 | 62.34 | 6.93 | 13.25 | 0.28 | 0.21 | 18.20 | 60.80 | 6.92 | 14.07 | 0.30 | 0.23 |
| 750 mM DT3 | 18.29 | 57.65 | 7.78 | 16.28 | 0.32 | 0.28 | 19.68 | 58.02 | 8.47 | 13.83 | 0.34 | 0.24 |
| 375 mM ED2 | 17.49 | 61.81 | 6.40 | 14.30 | 0.28 | 0.23 | 15.46 | 61.72 | 6.73 | 16.08 | 0.25 | 0.26 |
| 375 mM TT4 | 19.30 | 58.94 | 7.84 | 13.92 | 0.33 | 0.24 | 16.89 | 61.21 | 7.56 | 14.34 | 0.28 | 0.23 |
| 375 mM TP5 | 19.95 | 63.15 | 8.90 | 7.96 | 0.32 | 0.13 | 18.95 | 61.08 | 8.81 | 11.16 | 0.31 | 0.18 |
| 375 mM PH6 | 21.04 | 61.74 | 9.05 | 8.15 | 0.34 | 0.13 | 19.45 | 67.57 | 4.26 | 8.71 | 0.29 | 0.13 |

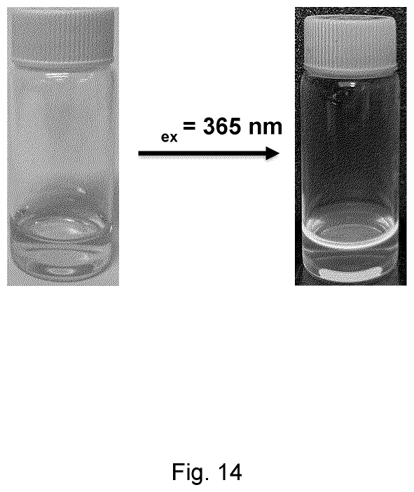

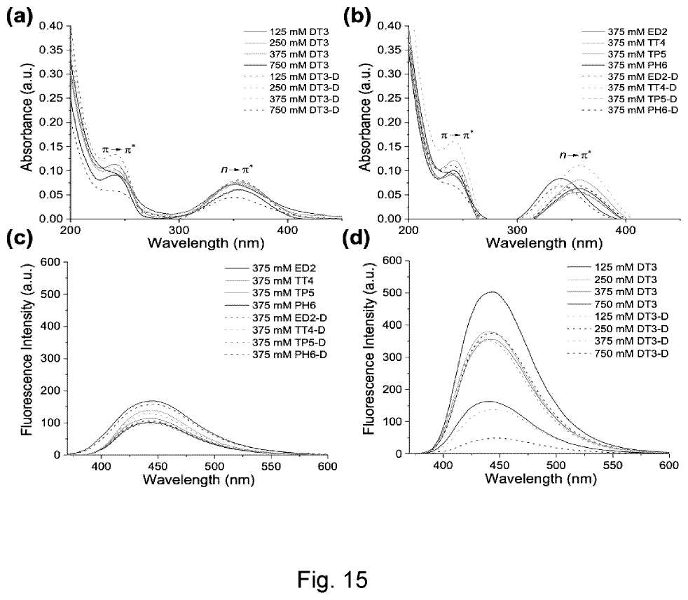

The optical properties of both protonated and deprotonated amine-passivated CDs were evaluated using UV-Vis and fluorescence spectroscopies. While the optical properties do not influence the catalytic properties, we confirm the formation of amine-passivated CDs using such techniques. Translucent yellow aqueous CD dispersions at concentrations of 75 μg mL⁻¹ exhibited bright blue fluorescence upon exposure to UV light at 365 nm (FIG. 14). For DT3-CDs, all CDs show two characteristic bands centred at 240 nm and 355 nm, regardless of their protonation form (FIG. 15a). The first absorbance band at 240 nm is attributed to the π→π* transition of the aromatic and alkene sp² domains of the carbon network.⁶⁴ The second band centred at 355 nm is assigned to the n→π* transitions of carboxyl and amide functional groups, primarily found on the surface of the CD structure.⁶⁵ TT4-, TP5- and PH6- passivated CDs show similar characteristic bands, attributed to the same π→π* and n→π* transitions (FIG. 15b). Interestingly, ED2-passivated CDs had their n→π* transition band centred at 335 nm, hypsochromic shifted in relation to the other amine-passivated CDs. It is hypothesized that the shift is caused by the decrease in nitrogenated groups. Indeed, the presence of nitrogen-containing substituents in sp²-hybdridized molecules has been shown to cause bathochromic and hyperchromic effects.⁶⁶

Furthermore, fluorescence (PL) spectra for all protonated and deprotonated CDs show similar fluorescence profiles, with a maximum fluorescence emission centred at 445 nm upon excitation at 350 nm, explaining the observed blue fluorescence (FIG. 15c,d). It was also observed that the emissions were excitation wavelength independent as the fluorescence maxima remains relatively unshifted with varying excitation wavelength. We note an expected decrease in emission intensity with changing wavelength as we excite away from the absorption maxima.

### Surface Properties of Amine-Passivated CDs

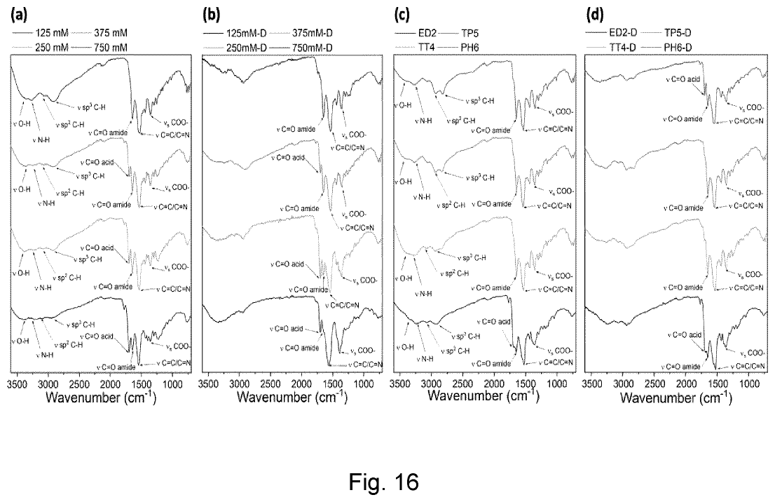

The surface of the CDs was extensively investigated using FT-IR spectroscopy as the surface functional groups play a crucial role in dictating the catalytic efficiency. Previous studies have shown that heterogeneous catalysts including CDs serve as contact catalysts, adsorbing reactants on their surfaces and directly catalysing the reaction.⁶⁷ DT3-CDs were synthesized using a concentration of DT3 from 125 mM to 750 mM as it has been reported that altering the concentration of DT3 will shift the number of carboxylic acids and amides fond on the surface of the CD.⁶⁸ As such, tailoring the surface functional groups is anticipated to provide key insights as to the governing catalysis mechanism for the transesterification reaction, focusing on the roles of functional groups such as carboxylates and amides. Furthermore, CDs were synthesized using 375 mM of various amine passivating agents (ED2, TT4, TP5, PH6) since the use of these precursors lead to the formation of CDs with differing oxygen to nitrogen ratios.⁶⁹ As expected, all the amine-passivated CDs (FIG. 16) also exhibit O—H and N—H stretching at ˜3380 cm⁻¹ and ˜3255 cm⁻¹, ascribed to the amine and alcohol moieties of the amine passivating agents and citric acid precursors, respectively.⁷⁰ The presence of carboxylic acids and amides is confirmed by the peaks at ˜1695 cm⁻¹ and ˜1645 cm⁻¹, attributed to carboxylic acid C═O and amide C═O stretching, respectively.⁶⁸ Previous studies attribute the formation of the amide bond to the reaction of carbonyl and amine groups within the precursors.⁷¹ The intense band at ˜1540 cm⁻¹ corresponds to C═C stretches of the skeletal polycyclic aromatic rings.⁷²

Varying the concentration of DT3 results in spectra that show a stark decrease in carboxylic acid C═O stretching and a corresponding increase in amide C═O stretching as the concentration of DT3 precursor increases (FIG. 16a); this trend has also been shown in a previous study, which confirms our results.⁶⁸ In the case of 750 mM DT3-CDs, the carboxylic C═O stretching is no longer observed. Furthermore, all CDs exhibited symmetric carboxylate stretching at ˜1360 cm⁻¹.⁶⁹ The presence of carboxylates before deprotonation is explained by the initial pH readings of the CDs (>pH 2.5), which are in close proximity of the pKa values (˜2.4) reported for carboxylic acids on CD surfaces.⁷³ Asymmetric carboxylate stretching is not observed due to the overlapping intense C═C/C═N stretching, which masks their presence. CDs passivated with ED2, TT4, TP5 and PH6 agents caused negligible changes to the surface chemistry for the most part when comparing to DT3-CDs, as illustrated by their FT-IR spectra in FIG. 16c; however, there were notable differences in amide and carboxylic C═O stretching, with a strong preference for amide formation. This is observed by the stronger transmittances for amide C═O stretching at ˜1645 cm⁻¹ relative to carboxylic acid C═O stretching at ˜1695 cm⁻¹. It should be noted that only protonated ED2- and DT3-CDs demonstrated carboxylic acid C═O stretching. Upon the addition of NaOH post-synthesis, 125 mM and 250 mM DT3-CDs show an increase in their symmetrical carboxylate stretching, intuitively attributed to the deprotonation of carboxylic acids by NaOH (FIG. 16b). Qualitatively, these amine-passivated CDs seem to have a higher surface concentration of carboxylic acids and had more acidic initial pH readings (pH ˜2.5) in relation to the other amine-passivated CDs. However, these vibrational features remained relatively consistent for the other amine-passivated CDs, including the 375 mM and 750 mM DT3-CDs (FIG. 16d); the lack of change in carboxylate stretching is attributed to their higher initial pH readings (>pH 5), where virtually all carboxylic acids are already deprotonated.

Since the surface potential of the catalyst is an important indicative of its mechanism of action, zeta-potential analysis (Table 4) was performed on protonated and deprotonated amine-passivated CDs to investigate their surface charges and their effect on biodiesel conversion.⁷⁴ All protonated DT3-CDs presented a slightly positive surface potential ranging from 0.5-9.5 mV. Differences in zeta-potential measurements are explained by the initial pH readings of their aqueous dispersions, where CDs with more acidic initial pH readings have higher zeta-potentials.⁷⁵ Protonated 375 mM amine-passivated CDs are considered approximately neutral, falling in the range of −4.5 to 1.5 mV. Therefore, prior to deprotonation, all CDs exhibit relatively neutral surface potentials. Upon deprotonation, as expected, the zeta-potential measurements for all CDs evidence negative surface potentials, falling in a range −4.5 to −30 mV. For the most part, a decrease in surface potential is accompanied by a concomitant increase in the surface concentration of carboxylic acid for the DT3-CDs.

Table 4 shows (a) Average zeta-potential and conductivity measurements of protonated and deprotonated DT3-CDs synthesized at 210° C. using 125-750 mM DT3; (b) Average zeta-potential and conductivity measurements of protonated and deprotonated amine-passivated CDs synthesized at 210° C. using 375 mM of different amine-passivating agents. All deprotonated CDs exhibit negative surface potentials.

TABLE 4
Zeta-potential analysis of the amine-passivated carbon dots

| | Protonated | | Deprotonated | |
|---|---|---|---|---|
| | Zeta- | | Zeta- | |
| Amine- | Potential | Conductivity | Potential | Conductivity |
| Passivated CDs | (mV) | (μS/cm) | (mV) | (μS/cm) |
| 125 mM DT3 | 7.4 | 0.52 | −29.6 | −2.10 |
| 250 mM DT3 | 9.4 | 0.66 | −26.0 | −1.85 |
| 375 mM DT3 | 0.5 | 0.04 | −16.1 | −1.14 |
| 750 mM DT3 | 1.4 | 0.10 | −20.0 | −1.42 |
| 375 mM ED2 | −4.5 | −0.32 | −10.3 | −0.73 |
| 375 mM TT4 | −2.2 | −0.16 | −5.2 | −0.37 |
| 375 mM TP5 | 0.3 | 0.02 | −4.7 | −0.33 |
| 375 mM PH6 | 1.2 | 0.08 | −14.2 | −1.01 |

### Quantification of Biodiesel Following Transesterification Reactions

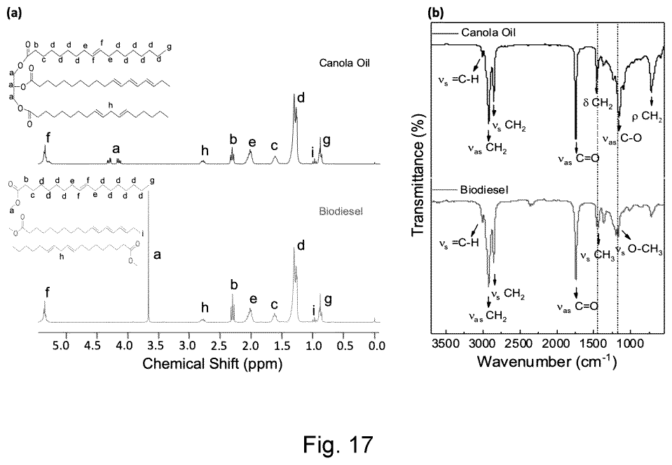

In the transesterification of vegetable oils, the reaction yield is characterized by the formation of fatty acid methyl esters (FAME) when using methanol as the alcohol.⁷⁶ Conventionally, FAME yields are quantified using gas-liquid chromatography and high-performance liquid chromatography, which usually require a high degree of accuracy during sample preparation and pre-treatment or sample derivatization.⁷⁷ However, ¹H NMR can also be used to accurately determine FAME conversion, avoiding the issues associated with other quantitative techniques. This technique exploits the differences in chemical shifts derived from the triglyceride precursor and the FAME product (FIG. 17a). The first difference arises from the multiplet at ˜4.30 ppm, representing the protons of the triglyceride backbone. As canola oil is converted to biodiesel, the multiplet disappears as expected. The second difference arises from the singlet at 3.66 ppm, representing the protons of the methoxy group in FAME, and has its relative peak intensity increase as biodiesel is formed. Since integrated areas are not absolute, the triplet at 2.30 ppm representing the α-methylene group in triglyceride derivatives and FAME was used as a basis point for calculations. A comparison of the integrated area from the singlet at 3.66 ppm relative to the triplet at 2.30 ppm can be used as an absolute indication of biodiesel conversion. Through the calibration curve constructed using pre-determined amounts of methyl esters and canola oil,³¹ the ratio between the integrations of peaks at 3.66 and 2.30 ppm enables the quantification of transesterification yields.⁷⁸ Furthermore, FT-IR analysis (FIG. 17b) can be used to qualitatively monitor biodiesel conversion. Both biodiesel and triglyceride functional groups exhibit cis sp² C—H stretching at ˜3010 cm⁻¹, asymmetrical and symmetrical methylene stretching at ˜2930 cm⁻¹ and ˜2860 cm⁻¹, respectively, as well as ester carbonyl stretching at ˜1,750 cm⁻¹.⁷⁹ However, significant changes are observed at ˜1450 cm⁻¹ and 1196 cm⁻¹, where the characteristic splitting exhibited by the asymmetric methyl and methoxy stretching peaks, respectively, are observed in biodiesel.⁸⁰

CD-Catalysed Transesterification of Canola Oil

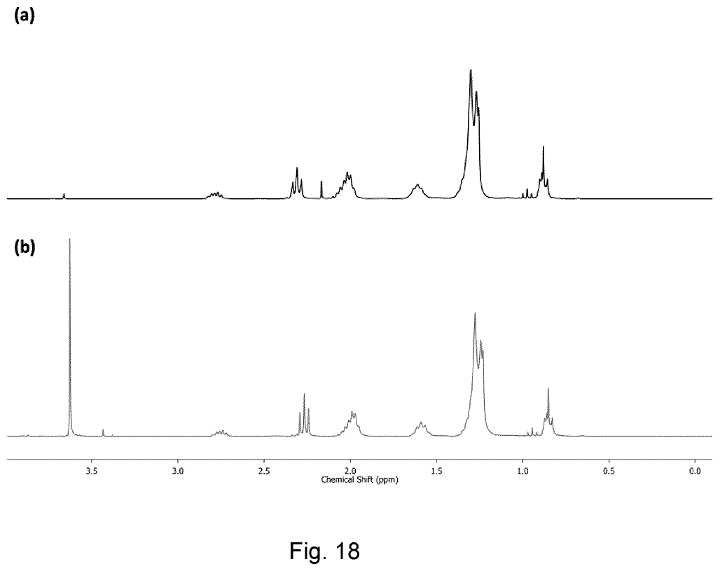

In optimizing the CD-catalysed transesterification of canola oil, altering reaction parameters such as catalyst loading, methanol content, temperature and reaction time were investigated. Heterogeneous base catalysts often require higher catalysts loadings, reaching up to 15 wt %, in order to have a comparable catalytic efficiencies similar to strong bases.⁸¹ Therefore, initial reaction mixtures were heated at 150° C. for 3 hours and protonated CDs were loaded at a 10 wt % content with an oil to methanol ratio of 1:72. A high oil to methanol molar ratio was used as the excess alcohol would drive the reaction to favour the methyl ester formation.⁸² Additionally, in comparison to conventional reaction temperatures of 70° C., a high reaction temperature was used since heterogeneous catalysts require energy intensive conditions to further drive the reaction forward.⁸³ Furthermore, a 10 wt % content was used to further drive the reaction forward since no signs of biodiesel formation were observed. However, even in using energy intensive conditions and a high methanol to oil ratio and catalyst loading, no biodiesel conversion was observed when using any of the protonated CD catalyst. A representative ¹H NMR spectrum of a protonated 750 mM DT3-CDs catalysed transesterification reaction (FIG. 18a) illustrates the negligible biodiesel conversion yield achieved by CDs in their protonated form, demonstrated by the lack of the methoxy singlet at 3.66 ppm. The observed results are expected due to the negligible number of active-site carboxylates on the surface of the CDs, which were hypothesized to be required for the CD-catalysed mechanism. Therefore, to catalyse the transesterification reaction using amine-passivated CDs, aqueous CD dispersions were deprotonated using NaOH to increase the number of carboxylate moieties present on the surface. Using the same reaction conditions as those used during protonated CD catalysis, the deprotonated CDs caused a drastic increase in biodiesel conversion (FIG. 18b); the 750 mM DT3-CDs even reached biodiesel conversion yields of 98% and were chosen for the subsequent optimization.

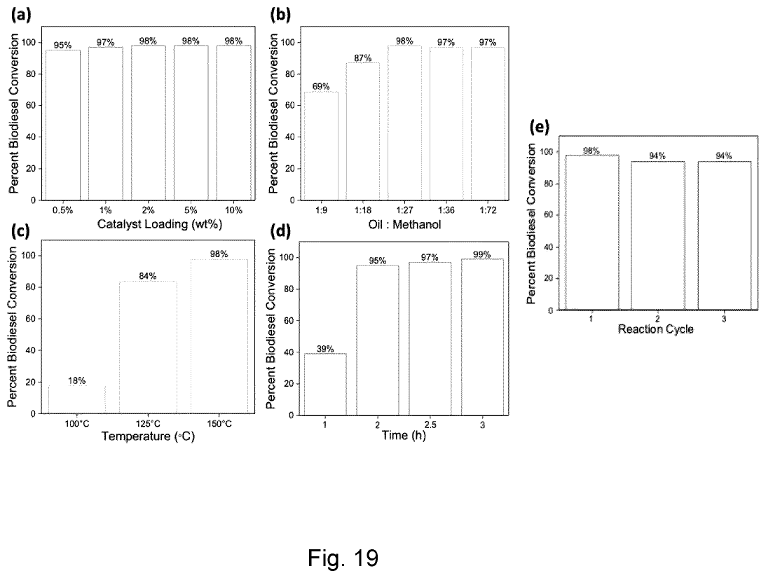

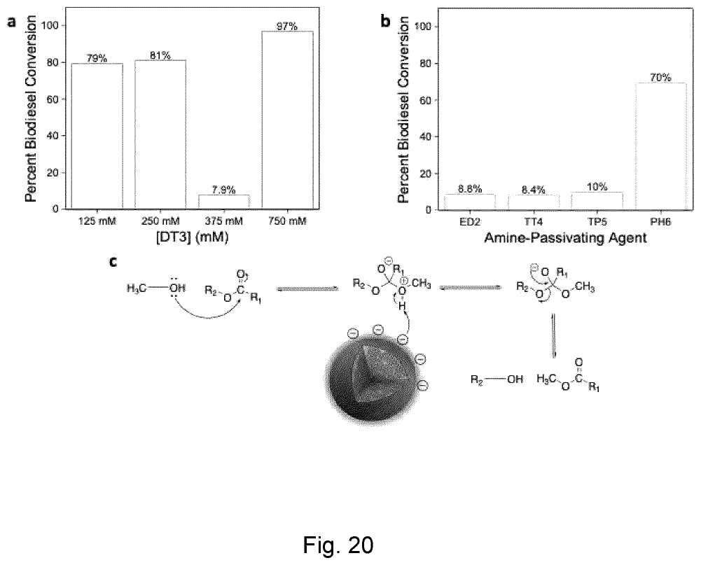

Thus, biodiesel optimization experiments were carried out using 750 mM DT3-CD catalyst loadings ranging from 0.5-10 wt % (FIG. 19a). All other reaction parameters were kept constant to investigate the effect of catalyst loading. Reactions were carried out using a 1:72 oil to methanol ratio and heated at 150° C. for 3 hours. At catalyst loadings of 0.5 wt % and higher, conversion yields of 95% are observed. Upon investigation of the effect of methanol, reactions with a 1 wt % catalyst loading exhibit significantly higher biodiesel conversions at an oil to methanol ratio of 1:36 in contrast to the reactions with a 0.5 wt % catalyst loading. Therefore, a catalyst loading of 1 wt % was selected for the ensuing optimization of methanol content. Reactions heated at 150° C. for 3 hours with a 1 wt % catalyst loading had their oil to methanol ratios varied from 1:9 to 1:72 (FIG. 19b). At a methanol to oil ratio of 1:27, the transesterification yields reach a plateau, achieving a biodiesel conversion of 98%. Following the optimization of the reaction's methanol content, the effects of altering reaction temperature were examined at a catalyst loading of 1% and a methanol content of 1:27 (FIG. 19c); the results show that a high reaction temperature of 150° C. was required to drive the reaction to an optimal conversion yield (>95%), where conversion yields were found to be significantly lower below 150° C. However, it was also observed that significant biodiesel conversion yields were achieved for reaction times of 2 hours and higher (FIG. 19d).

Following the optimization of the CDs, the reusability of the CDs in catalysing the transesterification reaction was investigated; the reusability of heterogeneous catalysts is one of the primary benefits in contrast to their homogeneous counterparts. Upon the completion of the reaction, the mixture was left in the oven at 80° C. overnight to allow for the evaporation of excess methanol. The mixture was then centrifuged, and the biodiesel phase was separated from the glycerol/CD phase. Following separation, the glycerol/CD phase was washed 3 times with hexanes to ensure the removal of any remaining biodiesel. The CD phase was then added to the reaction vessel and the subsequent transesterification reaction was carried out using the same conditions. As demonstrated by FIG. 19e, there is a slight decrease in biodiesel conversion after the 1st catalytic cycle. However, the conversion yield seems to plateau at a biodiesel conversion of 94% even after 3 catalytic cycles.

### Mechanism of the Amine-CD Catalysed Transesterification

In investigating the mechanism of biodiesel conversion, the catalytic efficiencies of all protonated and deprotonated amine-CDs were tested for their transesterification activity using canola oil and methanol as precursors. Furthermore, changes in surface chemistries and their effects on biodiesel conversion were explored by altering the type of amine passivating agent and their concentration used during synthesis. As previously mentioned, no biodiesel conversion was observed when using any of the protonated CDs due to the negligible number of active-site carboxylates on the surface of the CDs. Upon deprotonation, biodiesel conversion yields up to 97% are observed at a 1 wt % catalyst loading. Interestingly, unlike the hypothesized CD-catalysed mechanism proposed by Macina et al., the results indicate that the CD-catalysed mechanism is not reliant on carboxylates present on the surface of the CDs. As demonstrated by the FT-IR spectra, there is a significant decrease in symmetrical carboxylate stretching as the concentration of DT3 precursor increases; however, as indicated by the observed biodiesel conversions, the conversion yields increase as the concentration of DT3 precursor increases. Furthermore, deprotonated 750 mM DT3-CDs, which have the lowest number of carboxylates in relation to the other DT3-CDs, achieved the highest biodiesel yields, further indicating that carboxylates may not be the key catalytic factors in the CD-catalysed transesterification mechanism.

Since CDs can mimic homogeneous base catalysts, it can be postulated that a decrease in zeta-potential would cause an increase in biodiesel conversion by introducing carboxylate groups that can act as key catalytic factors in the transesterification mechanism. In accordance with the zeta-potential measurements, CDs with a higher concentration of nitrogenated surface species require a less negative surface potential to catalyse the reaction. For this reason, it is postulated that a negligible biodiesel conversion yield was observed for the 375 mM DT3-CDs. Due to the increase in oxygenated surface species when comparing to the 750 mM DT3-CDs, the 375 mM DT3-CDs required a larger negative surface charge to be catalytically efficient. This can be achieved through the further deprotonation of surface functional groups. The importance of the overall surface charge as a key factor in optimizing CDs is also demonstrated by the biodiesel conversion yields for the CDs synthesized using various amine-passivating agents. 375 mM PH6-CDs were the only CDs passivated at 375 mM to achieve significant yields and aside from the DT3-CDs, had the largest negative surface potential. Interestingly, as shown by previous elemental analyses of the CDs synthesized from the same precursors in the Naccache laboratory, PH6-CDs had the highest nitrogen to oxygen ratio in relation to the other amine-passivated CDs. This is further evidence that the mechanism is reliant on nitrogenated species on the surface of the CDs and that the overall surface charge plays an important catalytic role.

Without wishing to be bound by theory, it is hypothesized that the catalytic mechanism works in a similar way to one proposed by Macina et al. It was proposed that the methanol attacks the ester, forming a zwitterion intermediate; subsequently, there is a proton transfer from the zwitterion to functional groups on the surface of the CD followed by the traditional base-catalysed mechanism steps. However, it is hypothesized that the conjugate bases of secondary amides are the key catalytic factors rather than carboxylates, acting as weak bases able to deprotonate the zwitterion. It has been reported previously that the transesterification mechanism can occur with weak bases; however, it requires high pressures and reaction temperatures to overcome the energy barrier. Since biodiesel conversion was only observed at a temperature of 150° C. in the hydrothermal reactor (a high-pressure environment), this supports that weak bases are useful in the CD-catalysed mechanism. Furthermore, the presence of conjugate bases of secondary amides are supported in two-fold manner. Firstly, the presence of secondary amides is confirmed by FT-IR spectra. All amine-passivated CDs exhibit amide C═O stretching at ˜1645 cm⁻¹ and lack symmetric N—H stretching at 3180 cm⁻¹, indicating the presence of secondary amides on the surface of the CDs. Secondly, a negative surface potential was necessary in achieving a high transesterification activity, suggesting the key catalytic factors are negatively charged.

### CONCLUSION

Heterogeneous catalysts have several beneficial characteristics relative to their commercially used homogeneous counterparts, emerging as a potential cost-reducing and energy efficiency-increasing solution. They afford a cheaper and environmentally benign purification, the reusage of the catalyst over several catalytic cycles and the prevention of saponification. Here, it is demonstrated a heavy metal-free CD-catalysed transesterification of canola to biodiesel, achieving biodiesel conversions of ≥97% at using 1 wt % catalyst loading at 150° C. and 2-hour reaction temperature and time, respectively. Furthermore, their reusability is shown over at least 3 catalytic cycles, highlighting their catalytic efficiency and robustness.

Although several heterogeneous catalysts have been developed in the production of biodiesel, they generally require expensive materials and tedious synthesis processes, are prone to metal leaching due to their metal composition and generally necessitate a high catalyst loading to be catalytically efficient. The heterogeneous CD catalysts described in this paper offer several advantages over previously reported heterogeneous catalysts. Firstly, the precursors used to synthesize the CD catalysts are abundant and cheap, enabling the adoption of cost-reducing catalysts over several reaction cycles. Secondly, industry-standard biodiesel conversions were achieved using only a 1 wt % catalyst loading. Lastly, their heavy metal-free composition prevents the leaching of metal ions into the biodiesel. The production of inexpensive, effective and re-usable heterogeneous CD catalysts has the potential to enhance and improve current biodiesel production methods due to their significant cost-reducing and efficiency-increasing benefits.

### REFERENCES

1 A. F. Lee, J. A. Bennett, J. C. Manayil and K. Wilson, Chem. Soc. Rev., 2014, 43, 7887-7916.
2 H. Schobert, Chemistry of Fossil Fuels and Biofuels, Cambridge University Press, Cambridge, 2013.
3 N. Abas, A. Kalair and N. Khan, Futures, 2015, 69, 31-49.
4 R. Quadrelli and S. Peterson, Energy Policy, 2007, 35, 5938-5952.
5 R. Dones, T. Heck and S. Hirschberg, Encycl. Energy, 2004, 77-95.
6 A. E. Atabani, A. S. Silitonga, I. A. Badruddin, T. M. I. Mahlia, H. H. Masjuki and S. Mekhilef, Renew. Sustain. Energy Rev., 2012, 16, 2070-2093.
7 P B. K. Uprety, W. Chaiwong, C. Ewelike and S. K. Rakshit, Energy Conyers. Manag., 2016, 115, 191-199.
8 V. K. Mishra and R. Goswami, Biofuels, 2018, 9, 273-289.
9 U. Rashid and F. Anwar, Fuel, 2008, 87, 265-273.
10 S. Mandal and K. Kundu, Biofuels, 2019, 0, 1-7.
11 M. Agarwal, G. Chauhan, S. P. Chaurasia and K. Singh, J. Taiwan Inst. Chem. Eng., 2012, 43, 89-94.
12 E. Lotero, Y. Liu, D. E. Lopez, K. Suwannakarn, D. A. Bruce and J. G. Goodwin, Ind. Eng. Chem. Res., 2005, 44, 5353-5363.
13 J. Zhang and L. Jiang, Bioresour. Technol., 2008, 99, 8995-8998.
14 J. M. Marchetti, V. U. Miguel and A. F. Errazu, Renew. Sustain. Energy Rev., 2007, 11, 1300-1311.
15 G. Arzamendi, I. Campo, E. Arguiñarena, M. Sánchez, M. Montes and L. M. Gandia, Chem. Eng. J., 2007, 134, 123-130.
16 M. Zabeti, W. M. A. Wan Daud and M. K. Aroua, Fuel Process. Technol., 2009, 90, 770-777.
17 X. Liu, H. He, Y. Wang, S. Zhu and X. Piao, Fuel, 2008, 87, 216-221.
18 X. Liu, H. He, Y. Wang and S. Zhu, Catal. Commun., 2007, 8, 1107-1111.
19 E. M. Björk, M. P. Militello, L. H. Tamborini, R. Coneo Rodriguez, G. A. Planes, D. F. Acevedo, M. S. Moreno, M. Odén and C. A. Barbero, Appl. Catal. A Gen., 2017, 533, 49-58.
20 Y.-Y. Wang, H.-H. Wang, T.-L. Chuang, B.-H. Chen and D.-J. Lee, Energy Procedia, 2014, 61, 933-936.
21 R. Hartono, A. Wijanarko and H. Hermansyah, IOP Conf. Ser. Mater. Sci. Eng., 2018, 345, 012002.
22 A. Brito, M. E. Borges and N. Otero, Energy & Fuels, 2007, 21, 3280-3283.
23 C. Garcia-Sancho, R. Moreno-Tost, J. M. Mérida-Robles, J. Santamaria-González, A. Jiménez-López and P. Maireles-Torres, Appl. Catal. B Environ., 2011, 108-109, 161-167.
24 W. Xu, L. Gao, S. Wang and G. Xiao, Bioresour. Technol., 2014, 159, 286-291.
25 V. Brahmkhatri and A. Patel, Ind. Eng. Chem. Res., 2011, 50, 6620-6628.
26 H. C. Chen, H. Y. Ju, T. T. Wu, Y. C. Liu, C. C. Lee, C. Chang, Y. L. Chung and C. J. Shieh, J. Biomed. Biotechnol., 2011, 2011, 5-10.
27 X. Zhao, F. Qi, C. Yuan, W. Du and D. Liu, Renew. Sustain. Energy Rev., 2015, 44, 182-197.
28 M. K. Lam, K. T. Lee and A. R. Mohamed, Biotechnol. Adv., 2010, 28, 500-518.
29 S. Shah, S. Sharma and M. N. Gupta, Energy and Fuels, 2004, 18, 154-159.
30 L. J. Konwar, J. Boro and D. Deka, Renew. Sustain. Energy Rev., 2014, 29, 546-564.
31 T. N. Pham-Truong, T. Petenzi, C. Ranjan, H. Randriamahazaka and J. Ghilane, Carbon N. Y., 2018, 130, 544-552.
32 N. C. T. Martins, J. Ângelo, A. V. Girão, T. Trindade, L. Andrade and A. Mendes, Appl. Catal. B Environ., 2016, 193, 67-74.
33 H. Yu, R. Shi, Y. Zhao, G. I. N. Waterhouse, L.-Z. Wu, C.-H. Tung and T. Zhang, Adv. Mater., 2016, 28, 9454-9477.
34 S. Sarkar, K. Das and P. K. Das, Langmuir, 2016, 32, 3890-3900.
35 T. Shen, Q. Wang, Z. Guo, J. Kuang and W. Cao, Ceram. Int., 2018, 44, 11828-11834.
36 Y. Wang, X. Chang, N. Jing and Y. Zhang, Anal. Methods, 2018, 10, 2775-2784.
37 H. Ding, S.-B. Yu, J.-S. Wei and H.-M. Xiong, ACS Nano, 2016, 10, 484-491.
38 J. Manioudakis, F. Victoria, C. Thompson, L. Brown, M. Movsum, R. Lucifero and R. Naccache, J. Mater. Chem. C, 2018, 7, 1-14.
39 Y. Chen, H. Lian, Y. Wei, X. He, Y. Chen, B. Wang, Q. Zeng and J. Lin, Nanoscale, 2018, 10, 6734-6743.
40 S. B. A. Hamid and R. Schloegl, ChemInform, 2005, 36, 1688-1692.
41 S. Z. Ali Ahamed, G. R. Dillip, P. Raghavaiah, K. Mallikarjuna and B. Deva Prasad Raju, Arab. J. Chem., 2013, 6, 429-433.
42 P. Pimpang, R. Sumang and S. Choopun, Chiang Mai J. Sci., 2018, 45, 2005-2014.
43 C. López, M. Zougagh, M. Algarra, E. Rodriguez-Castellón, B. B. Campos, J. C. G. Esteves Da Silva, J. Jiménez-Jiménez and A. Rios, Talanta, 2015, 132, 845-850.
44 H. M. Mahmudul, F. Y. Hagos, R. Mamat, A. A. Adam, W. F. W. Ishak and R. Alenezi, Renew. Sustain. Energy Rev., 2017, 72, 497-509.
45 M. MONTEIRO, A. AMBROZIN, L. LIAO and A. FERREIRA, Talanta, 2008, 77, 593-605.
46 I. M. Atadashi, M. K. Aroua and A. A. Aziz, Renew. Energy, 2011, 36, 437-443.
47 G. Gelbard, O. Bras, R. M. Vargas, F. Vielfaure and U. F. Schuchardt, J. Am. Oil Chem. Soc., 1995, 72, 1239-1241.
48 G. Knothe, 1999, 76, 795-800.
49 F. Ma and M. A. Hanna, Bioresour. Technol., 1999, 70, 1-15.
50 L. C. Meher, D. V. Sagar and S. N. Naik, 2006, 10, 248-268.
51 S. H. Teo, Y. H. Taufiq-Yap, U. Rashid and A. Islam, RSC Adv., 2015, 5, 4266-4276.
52 A. P. S. Chouhan and A. K. Sarma, Renew. Sustain. Energy Rev., 2011, 15, 4378-4399.
53 M. Di Serio, R. Tesser, L. Pengmei and E. Santacesaria, 2008, 207-217.
54 D. F. Taber, J. C. Amedio and Y. K. Patel, J. Org. Chem., 1985, 50, 3618-3619.
55 G. Höfle, W. Steglich and H. Vorbrüggen, Angew. Chemie Int. Ed. English, 1978, 17, 569-583.
56 J. Yao, L. Ji, P. Sun, L. Zhang and N. Xu, Fuel, 2010, 89, 3871-3875.
57 U. Schuchardt, R. Sercheli and R. Matheus, J. Braz. Chem. Soc., 1998, 9, 199-210.
58 M. Sirajuddin, M. Tariq and S. Ali, J. Organomet. Chem., 2015, 779, 30-38.
59 A. Macina, T. V. De Medeiros and R. Naccache, J. Mater. Chem. A, 2019, 7, 23794-23802.
60 K. M. Tripathi, T. S. Tran, T. T. Tung, D. Losic and T. Kim, J. Nanomater., DOI:10.1155/2017/7029731.
61 A. S. Rettenbacher, B. Elliott, J. S. Hudson, A. Amirkhanian and L. Echegoyen, Chem.—A Eur. J., 2005, 12, 376-387.
62 T. T. Meiling, R. Schümann, S. Vogel, K. Ebel, C. Nicolas, A. R. Milosavljević and I. Bald, J. Phys. Chem. C, 2018, 122, 10217-10230
63 A. B. Bourlinos, R. Zbořil, J. Petr, A. Bakandritsos, M. Krysmann and E. P. Giannelis, Chem. Mater., 2012, 24, 6-8.
64 D. Pan, J. Zhang, Z. Li and M. Wu, Adv. Mater., 2010, 22, 734-738.
65 W. F. Zhang, H. Zhu, S. F. Yu and H. Y. Yang, Adv. Mater., 2012, 24, 2263-2267.
66 K. Bowden, E. A. Braude and E. R. H. Jones, J. Chem. Soc., 1946, 948-952.
67 W. Huang and W. X. Li, Phys. Chem. Chem. Phys., 2019, 21, 523-536.
68 J. Manioudakis, F. Victoria, C. A. Thompson, L. Brown, M. Movsum, R. Lucifero and R. Naccache, J. Mater. Chem. C, 2019, 7, 853-862.
69 M. Zheng, S. Liu, J. Li, Z. Xie, D. Qu, X. Miao, X. Jing, Z. Sun and H. Fan, J. Mater. Res., 2015, 30, 3386-3393.
70 L. Cheng, Y. Li, X. Zhai, B. Xu, Z. Cao and W. Liu, ACS Appl. Mater. Interfaces, 2014, 6, 20487-20497.
71 S. Hill and M. C. Galan, Beilstein J. Org. Chem., 2017, 13, 675-693.
72 V. lucureanu, A. Matei and A. M. Avram, Crit. Rev. Anal. Chem., 2016, 46, 502-520.
73 K. Syamantak, C. V. Navneet, G. Prashant, J. Sanjhal, G. Souvik and C. K. Nandi, J. Mater. Sci. Eng., 2018, 07, 1000448.
74 Y. Bai, H. Huang, C. Wang, R. Long and Y. Xiong, Mater. Chem. Front., 2017, 1, 1951-1964.
75 S. Ferraris, M. Cazzola, V. Peretti, B. Stella and S. Spriano, Front. Bioeng. Biotechnol., 2018, 6, 1-7.
76 D. J. Anneken, S. Both, R. Christoph, G. Fieg, U. Steinberner and A. Westfechtel, Ullmann's Encycl. Ind. Chem., 2012, 14, 73-116.
77 P. De Filippis, C. Giavarini, M. Scarsella and M. Sorrentino, J. Am. Oil Chem. Soc., 1995, 72, 1399-1404.
78 G. Gelbard, O. Bras, R. M. Vargas, F. Vielfaure and U. F. Schuchardt, J. Am. Oil Chem. Soc., 1995, 72, 1239-1241.
79 M. Tariq, S. Ali, F. Ahmad, M. Ahmad, M. Zafar, N. Khalid and M. A. Khan, Fuel Process. Technol., 2011, 92, 336-341.
80 S. N. Rabelo, V. P. Ferraz, L. S. Oliveira and A. S. Franca, Int. J. Environ. Sci. Dev., 2015, 6, 964-969.
81 S. Ramu, N. Lingaiah, B. L. A. Prabhavathi Devi, R. B. N. Prasad, I. Suryanarayana and P. S. Sai Prasad, Appl. Catal. A Gen., 2004, 276, 163-168.
82 M. Zabeti, W. M. A. Wan Daud and M. K. Aroua, Fuel Process. Technol., 2009, 90, 770-777.
83 X. Liu, H. He, Y. Wang and S. Zhu, Catal. Commun., 2007, 8, 1107-1111.

1. Use of carbon dots to catalyze a transesterification reaction, wherein the transesterification is carried out at a temperature of about 50° C. to about 250° C.

2-7. (canceled)

8. The use of claim 1, wherein the transesterification is carried out at a temperature of about 125° C. to about 175° C.

9. The use of claim 1, wherein the transesterification is carried out at a temperature of about 140° C. to about 160° C.

10. The use of claim 1, wherein the transesterification is carried out at a temperature of about 150° C.

11. The use of claim 1, wherein the transesterification is carried out with a catalyst loading of about 0.1 to about 40 wt %.

12. The use of claim 1, wherein the transesterification is carried out with a catalyst loading of about 0.2 to about 20 wt %.

13. The use of claim 1, wherein the transesterification is carried out with a catalyst loading of about 0.5 to about 10 wt %.

14. The use of claim 1, wherein the transesterification is carried out with a catalyst loading of about 0.8 to about 1.5 wt %.

15. The use of claim 1, wherein the transesterification is carried out with a catalyst loading of about 1.0 wt %.

16. The use of claim 1, wherein the transesterification is carried out in an oil:alcohol molar ratio of about 1:5 to about 1:200.

17. The use of claim 1, wherein the transesterification is carried out in an oil:alcohol molar ratio of about 1:40 to about 1:80.

18. The use of claim 1, wherein the transesterification is carried out in an oil:alcohol molar ratio of about 1:60.

19. The use of claim 1, wherein the transesterification is carried out in an oil:molar ratio of about 1:15 to about 1:35.

20. The use of claim 1, wherein the transesterification is carried out in an oil:alcohol molar ratio of about 1:25 to about 1:30.

21. The use of claim 16, wherein the alcohol is chosen from methanol, ethanol, butanol, propanol, iso-propanol, and mixtures thereof.

22. The use of claim 1, wherein the carbon dots are selected from glycine-citric acid carbon dots (GlyCDs), amine-passivated carbon dots (amine-passivated CDs), and combinations thereof.

23-217. (canceled)
